(19) United States
(12) Patent Application Publication
(10) Pub. No.: US 2026/0000789 A1
(43) Pub. Date: Jan. 1, 2026

(54) BAG3 METHODS AND USES FOR TREATMENT OF INFLAMMATION

(71) Applicants: TEMPLE UNIVERSITY OF THE COMMONWEALTH SYSTEM OF HIGHER EDUCATION, Philadelphia, PA (US); LOYOLA UNIVERSITY OF CHICAGO, Maywood, IL (US)

(72) Inventors: Arthur M. FELDMAN, Wynnewood, PA (US); Jonathan KIRK, Maywood, IL (US)

(73) Assignees: TEMPLE UNIVERSITY OF THE COMMONWEALTH SYSTEM OF HIGHER EDUCATION, Philadelphia, PA (US); LOYOLA UNIVERSITY OF CHICAGO, Maywood, IL (US)

(21) Appl. No.: 18/703,672

(22) PCT Filed: Oct. 21, 2022
(86) PCT No.: PCT/US2022/047479

§ 371 Date: Apr. 22, 2024

Related U.S. Application Data
(60) Provisional application No. 63/262,953, filed on Oct. 22, 2021.
(60) Provisional application No. 63/368,765, filed on Jul. 18, 2022.

(51) Int. Cl. A61K 48/00; A61K 38/17; A61P 37/06; C12N 15/86

(52) U.S. Cl. CPC A61K 48/0058; A61K 38/1709; A61K 48/0083; A61P 37/06; C12N 15/86; C12N 2750/14143

(57) ABSTRACT

Bag3 is a multifunctional protein expressed predominantly in the heart, the skeletal muscle, the central nervous system and in many cancers. Although BAG3 was cloned only a decade ago, studies have shown that genetic variants, particularly those that result in haplo-insufficiency, can lead to severe left ventricular dysfunction; however, the full mechanisms responsible have remained obscure. To obviate the influence of heart failure itself on the biology of Bag3, transgenic mice harboring a single allele knock-out were studied between 8 and 10 weeks of age before any obvious signs of heart failure were evident. The results were surprising and informative. First, it was found that despite a normal phenotype, young Bag3^{+/-} had marked changes in the proteome that were characterized by changes in proteins associated with metabolism and apoptosis. Consistent with this finding, a decrease in the levels of critical proteins charged with maintaining the mitochondrial membrane potential was observed. It was also found that young mice shifted from a balance between the extrinsic and intrinsic pathways of apoptosis. However, in the presence of stress and the absence of Bag3 there was a shift from a balanced to an extrinsic dominant system (cleaved caspase 8). The diverse array of critical pathways regulated by Bag3 suggests a more important role especially during stress and that this role might include serving as an intracellular glue that holds proteins where they can be most effective rather than having them meet accidentally.

### CROSS-REFERENCE TO RELATED APPLICATIONS

This patent application is the National Phase of International Application No. PCT/US2022/047479, filed Oct. 21, 2022, which designated the U.S. and that International Application was published under PCT Article 21(2) in English, which claims the benefit of priority to U.S. Provisional Application No. 63/262,953, filed Oct. 22, 2021, and U.S. Provisional Application No. 63/368,765, filed Jul. 18, 2022. The entire contents of the foregoing applications are incorporated herein by reference, including all text, tables, sequence listing and drawings

### GOVERNMENT SUPPORT

This invention was made with government support under HL 091799 and HL 123093 awarded by the National Institutes of Health. The government has certain rights in the invention.

### INTRODUCTION

BAG3 is a multifunctional protein that is expressed ubiquitously, but is most prominent in the heart, the skeletal muscles, the central nervous system and in many cancers (1,2). Multiple genome-wide association studies and whole exome or whole genome sequencing of DNA from patients with both hereditary and sporadic dilated cardiomyopathy (DCM) have shown that loss of a single allele of BAG3 is an important cause of disease (3-6). This has been confirmed in animal models as a homozygous deletion of BAG3 is lethal in the early post-natal period (7), whereas loss of function of a single allele leads to the development of myocyte dysfunction in zebrafish (3), in mesenchymal stem cell-derived cardiac myocytes carrying a human mutation (8), in mouse models with both point mutations and haplo-insufficiency (9,10), and in humans with haplo-insufficiency (11, 12). Studies have also shown that patients with heart failure and a reduced ejection fraction (HFrEF) who are genotypically normal have a reduction in BAG3 in the ventricular myocardium at the time of heart transplant that is comparable to that seen in patients with BAG3 truncations (11,12). In contrast with the heart, the over-expression of BAG3 in cancer cells results in chemotherapy resistance and an increased proclivity to metastasis and local invasion (13,14). Despite its obvious importance in the two leading diseases in the industrialized world, heart disease and cancer, the full scope of BAG3's role in health and disease has not been fully defined.

The structure of BAG3 has numerous protein-protein binding domains that allow it to influence a diverse array of molecular and cellular activities. In the heart, BAG3 augments autophagy by serving as a chaperone to heat shock protein 70 (hsp/hsc 70).(15) It inhibits apoptosis by interacting with the anti-apoptosis protein Bcl-2 (16), improves excitation-contraction coupling by linking the beta-adrenergic receptor (b-AR) and the L-type Ca²⁺ channel (17), and maintains the integrity of the sarcomere (18). The varying functions of BAG3 are facilitated by the presence of multiple binding sites and a diverse group of binding partners. For example, a PXXP domain serves as a molecular anchor for the proximal end of the motor-dynein transport system whereas two isoleucine-proline-valine (IPV) motifs bind the small heat shock proteins HspB6 and HspB8 and support macro-autophagy (1).

Recent studies in animal models have begun to link specific heterozygous genetic variants in BAG3 with unique cellular or molecular phenotypes. For example, the E455K loss of function mutation disrupts the interaction between BAG3 and Hsp-70 resulting in instability of the heat shock proteins and a loss in protein homeostasis (9). The rare P209L variant is a dominant gain of function mutation that causes aggregation of itself with Hsp70 clients which results in stalling of the Hsp70 autophagic network leading to a restrictive cardiomyopathy (19). By contrast, a P209S variant of BAG3 has been reported in association with a late onset axonal Charcot-Marie-Tooth neuropathy in two patients (20). However, a lack of clarity still exists as a result of inconsistencies in the effects of BAG3^{P209L} mutations on the phenotype of the murine heart (21)(22).

### SUMMARY

As disclosed herein, to gain a better understanding of the cardiac biology of BAG3 haplo-insufficiency high pressure liquid chromatography coupled to tandem mass spectrometry (LC-MS/MS) was used to identify proteins that are differentially expressed in young 8-10-week-old mice with a heterozygous BAG3 ablation: a time when left ventricular (LV) function and cardiac size are still normal. Mice with heterozygous knock-out of BAG3 are phenotypically normal by echocardiography at 8 to 10 weeks of age but demonstrate marked changes in left ventricular function by 18 weeks of age.

Proteomic analysis revealed two areas of cardiac biology: mitochondrial function and programmed cell death or apoptosis. Evaluation of the proteome of 8 to 10 week old BAG3^{+/-} mice revealed abnormalities in proteins associated with metabolism and programmed cell death or apoptosis despite a normal phenotype.

As disclosed herein, inter alia, it has now been discovered that BCL2-Associated Athanogene 3 (BAG3) is a critical component of both the intrinsic and extrinsic pathways of apoptosis in the heart and in other tissues and cell types. In particular, BAG3 regulates cellular apoptosis both through canonical as well as non-canonical pathways.

As also disclosed herein, inter alia, it has now been discovered that BAG3 modifies a late step in apoptosis, namely, the activation of caspase 3. BAG3 interacts directly with the inhibitor of apoptosis protein one/two (cIAP1/2 or cIAP). Under normal conditions, BAG3 protein is present in normal levels and binds to Cellular Inhibitor of Apoptosis 1 (CIAP-1), facilitating CIAP1's ability to bind to and inhibit activation of caspase 3. However, under conditions of BAG3 haploinsufficiency, there are marked abnormalities in mitochondrial function including a decrease in the membrane potential which leads to the Second Mitochondria-derived Activator of Caspase, or SMAC (also referred to as Direct IAP Binding Protein (DIABLO)), leaking out of the mitochondria. SMAC then translocates to the cytoplasm and binds CLAP-1, thereby releasing it from caspase 3 and allowing caspase 3 to be activated. Activated caspase 3 can then lyse key components of the cell. Accordingly, BAG3 can be used or formulated to reduce, inhibit or decrease activation of caspase-3, thereby reducing inflammation or an inflammatory response.

As further disclosed herein, it has now been discovered that decreased levels of BAG3 cause a shift from a balance between the intrinsic and extrinsic pathways of caspase activation to signaling that favors activation of caspase-8 (cleaved caspase 8). Accordingly, BAG3 can be used or formulated to reduce, inhibit or decrease activation of caspase-8.

As additionally disclosed herein, inter alia, it has now been discovered that BAG3 interacts directly with the mitochondrial import receptor subunit TOM22, and the Ca²⁺ uniporter, mitochondrial metabolism and the generation of the mitochondrial membrane potential respectively.

As additionally disclosed herein, inter alia, it has now been discovered that decreased BAG3 results in an increase in poly (ADP-ribose) polymerase 1 (PARP1). Such an increase in PARP1 can result in an increase in alpha-synuclein and a worsening of Parkinson's symptoms. Accordingly, BAG3 can be used or formulated to reduce, inhibit or decrease PARP1.

In accordance with the invention, BAG3 can be used or formulated to modulate TNF signaling. In particular embodiments, BAG3 can be used or formulated to reduce, inhibit or decrease TNF signaling.

In accordance with the invention, BAG3 can be used or formulated to modulate inflammation. In particular embodiments, BAG3 can be used or formulated to reduce, inhibit, decrease or treat inflammation.

In accordance with the invention, BAG3 can be used or formulated to modulate an inflammatory response. In particular embodiments, BAG3 can be used or formulated to reduce, inhibit, decrease or treat an inflammatory response.

The inflammation or inflammatory response can be systemic, regionally or locally, such as in an organ or tissue. In particular aspects, nonlimiting examples of inflammation or inflammatory response that BAG3 can be used or formulated to reduce, inhibit, decrease or treat occur in the pulmonary system, lung, cardiovascular system, central nervous system, bone, skeletal joints, skeletal muscle, gastrointestinal system, stomach, small intestine, large intestine, liver, kidney and pancreas.

In particular aspects, nonlimiting examples of inflammation or inflammatory response that BAG3 can be used or formulated to reduce, inhibit, decrease or treat include chronic inflammatory disease, chronic inflammatory demyelinating polyneuropathy, primary immune thrombocytopenia, geriatric anorexia, gut inflammation, inflammatory bowel disease, ulcerative colitis, Crohn's disease, lupus, rheumatoid arthritis, chronic myocarditis, chronic myocarditis after Covid 19 infection, psoriasis, psoriatic arthritis and ankylosing spondylitis.

In accordance with the invention, BAG3 can be used or formulated to modulate PARP1 levels, expression or activity. In particular embodiments, BAG3 can be used or formulated to reduce, inhibit, or decrease PARP1 levels, expression or activity. In particular embodiments, BAG3 can be used or formulated to reduce, inhibit, decrease or stabilize amounts of alpha-synuclein. In particular embodiments, BAG3 can be used or formulated to reduce, inhibit, decrease or decrease worsening or severity one or more symptoms of Parkinson's disease.

In particular embodiments, a BAG3 encoding nucleic acid comprises an expression vector expressing a BAG3 protein or active BAG3 peptide thereof.

In particular embodiments, an expression vector comprises a promoter, the promoter comprising an inducible promoter, a constitutive promoter, bicistronic promoter, tissue specific promoter or cardiac specific promoter.

In particular embodiments, an expression vector comprises a viral vector, cardiotropic vector, plasmid, or a yeast vector.

In particular embodiments, a viral or cardiotropic vector comprises an adenovirus vector, an adeno-associated virus vector (AAV), a coxsackie virus vector, cytomegalovirus vector, Epstein-Barr virus vector, parvovirus vector, or hepatitis virus vectors.

In particular embodiments, an AAV vector comprises a capsid protein having 90% or more sequence identity to any of AAV1, AAV2, AAV3, AAV4, AAV5, AAV6, AAV7m AAV8, AAV9, AAV10, AAV11 or AAV12.

In particular embodiments, an expression vector is a pseudotyped viral vector.

In particular embodiments, the inflammation or inflammatory response is induced or increased by a cytokine.

In particular embodiments, a cytokine comprises tumor necrosis factor (TNF).

In particular embodiments, a patient expresses lower than normal levels of BAG3 in a tissue or organ or does not detectably express or produce functional BAG3.

In particular embodiments, the inflammation or inflammatory response occurs in the pulmonary system, lung, cardiovascular system, central nervous system, bone, skeletal joints, skeletal muscle, gastrointestinal system, stomach, small intestine, large intestine, liver, kidney or pancreas.

In particular embodiments, an expression vector further comprises a promoter, the promoter optionally comprising an inducible promoter, a constitutive promoter, bicistronic promoter or tissue specific promoter.

In particular embodiments, a promoter confers expression in the pulmonary system, lung, cardiovascular system, central nervous system, bone, skeletal joints, skeletal muscle, gastrointestinal system, stomach, small intestine, large intestine, liver, kidney or pancreas.

In particular embodiments, an expression vector further comprises an AAV inverted terminal repeat (ITR).

In particular embodiments, an expression vector further comprises a polyadenylation sequence and/or stop codon.

In particular embodiments, a patient or subject is human.

In particular embodiments, a patient, subject or human has a mutation in their endogenous BAG3 polynucleotide or polypeptide.

In particular embodiments, a patient, subject or human has reduced expression or activity of endogenous BAG3 polynucleotide or polypeptide.

In particular embodiments, a viral vector is administered or formulated at a dose from about 0.1×10¹² vector genomes (vg)/weight of the patient in kilograms (vg/kg) to about 1.0×10¹⁴ vg/kg.

In particular embodiments, a viral vector is administered or formulated at a dose from about 1.0×10¹² vg/kg to about 0.5×10¹⁴ vg/kg.

In particular embodiments, a viral vector is administered or formulated at a dose from about 3.0×10¹² vg/kg to about 1.0×10¹³ vg/kg.

In particular embodiments, a viral vector is administered or formulated at a dose from about 3.0×10¹² vg/kg to about 9.0×10¹² vg/kg.

In particular embodiments, a viral vector is administered or formulated at a dose from about 3.0×10¹² vg/kg to about 8.0×10¹² vg/kg.

In particular embodiments, a viral vector is administered or formulated at a dose from about 3.0×10¹² vg/kg to about 5.0×10¹² vg/kg.

### DESCRIPTION OF DRAWINGS

FIGS. 1A-1B show data indicating that young mice with a deletion of one allele of Bag3 demonstrate a unique proteome that emphasizes alterations in the pathways of apoptosis and cellular metabolism despite the presence of normal LV function and size. A) Scatter plots with bar graphs showing results of transthoracic echocardiography of 8 to 10 wk old BAG3^{+/-} and BAG3^{wt} mice including measures of left ventricular (LV) ejection fraction (EF %), LV internal diameter in diastole (LVIDd) and LV internal diameter in systole (LVIDs) both in millimeters (mm). B) Levels of BAG3 were diminished by approximately 50% in all BAG3^{+/-} groups

FIGS. 2A-2B show data indicating that cardiomyocyte-specific Bag3 knockout (KO) disrupts expression of mitochondrial proteins involved in cell metabolism and apoptosis. (A) Volcano plot analysis of all proteins from the wild-type and BAG3-KO mouse left ventricle identified by bottom-up mass spectrometry; significance cut-off set to p=0.05; green indicates decreased expression compared with wild-type and red indicates increased expression; n=3 WT, 3 KO. B) Graphical summary of the proteins with altered expression the BAG3 KO mice grouped by their primary cellular compartment. C) Graphical summary of the proteins with altered expression in the BAG3 KO mice grouped by their biological function(s); biological function and cellular component information was obtained using the DAVID bioinformatics program (Version 6.8).

FIGS. 3A-3E show that TUNEL positive cells are higher but mitochondrial membrane potential are lower in Bag3^{+/-} mice subjected to hypoxia/reoxygenation. A) and B) TUNEL Staining. Adult mouse cardiomyocytes isolated from WT and BAG3^{+/-} mice were subjected to hypoxia/reoxygenation (H/R) as well as normoxia (Norm) control conditions. Cells were stained with Nonyl Acridine Orange (NAO), TMR red, and DAPI before imaging with a Zeiss LSM 900 confocal microscope. Statistical significance was determined using 1-way ANOVA with Bonferroni correction for multiple sub-comparisons. **** indicates <0.0001, “ns” indicates non-significant. n=10 images per group. Scale bar=50 μm. “C) Bcl-2 levels were not different in BAG3^{+/-} LV myocardium when compared with WT controls. D) MitoSOX Staining. Adult mouse cardiomyocytes isolated from WT and Bag3^{-/-} mice were stained with MitoSOX and imaged with a Zeiss LSM 900 confocal microscope. MitoSOX fluorescence is quantified using the Fiji Image J, and data are plotted in GraphPad Prism 7 software. Statistical significance was determined using t-test. “ns” indicates non-significant. n=75 to 182 cells per group. Scale bar=20 μm. E) TMRM Staining. Adult mouse cardiomyocytes isolated from WT and Bag3^{-/-} mice were stained with TMRM and imaged with a Zeiss LSM 900 confocal microscope. TMRM fluorescence is quantified using the Fiji Image J, and data are plotted in GraphPad Prism 7 software. Mitochondrial content is quantified as individual mitochondria area using the Mito-Morphology mjciacro in Image J. Statistical significance was determined using a t-test. ** indicates <0.001, “ns” indicates non-significant. n=136 to 162 cells per group. Scale bar=20 μm.

FIGS. 4A-4F show a Western blot analysis of proteins involved in mitochondrial dependent or mitochondrial independent apoptotic signaling in both young and old Bag3^{+/-} and Bag3^{WT} mice. Data shown in the individual bar graphs are derived from the accompanying western blots. The “n” was equal to 5 in each study group with the exception of the samples obtained from the older (18 week) mice in FIG. 3B where the sample size was 3 per investigational group. Each study was repeated at least once with tissue obtained from the same mice or identically aged mice. A) Levels of total caspase 3 are significantly (p<0.01) higher in BAG3^{-/-} mice than in BAG3^{WT} mice. B), the ratio of cleaved caspase 3 to pro caspase 3 in tissue from BAG3^{WT} mice was higher in BAG3^{WT} mice than in BAG3^{+/-} mice and the same was true in older 18 week-old mice despite the fact that LV EF was significantly diminished and LV dilatation was clear in the older mice (See FIG. 1A). C) Levels of TNFa are significantly elevated in BAG3^{-/-} mice but there is no change in levels of IL-6, suggesting that the cytokine effect is highly specific. D) By contrast with caspase 3, there was a significant (p<0.01) increase in levels of cleaved caspase 8 divided by total caspase 8, suggesting that caspase 8 was physiologically increased in mice with no obvious heart failure. E) A significant decrease (p<0.01) in the level of TOM22 was observed, a member of the TOM (translocase of the outer membrane) family of proteins that carry amino acids and small peptide sequences and membrane fragments across the mitochondrial membrane for subsequent incorporation into larger proteins produced in the mitochondrial matrix. F) shows immune-precipitation studies that sought to identify BAG3 partners in order to better understand the biology of BAG3 haplo-insufficiency. BAG3 bound TOM22 and cIAP but not the homologous XIAP or SMAC.

FIGS. 5A-5C show data indicating that neither SMAC nor cIAP are differentially expressed in mice with BAG3 haplo-insufficiency; however, SMAC requires Bag3 to translocate from the OMM (outer mitochondrial membrane) to the cytoplasm. A) Neonatal mouse ventricular myocytes (NMVM) were isolated and cultured under normoxic conditions, under normoxic conditions with one hour of hypoxia and two hours of normoxia, under normoxia but in the presence of an SiRNA for BAG3 and under normoxic experimental condition of hypoxia followed by reoxygenation. TOM22, (mitochondria) MCU (mitochondria) and GAPDH (cytoplasm) were used as controls to demonstrate that separation of the mitochondria had been accomplished. BAG3 was expressed to a greater degree in the cytoplasm of H/R-stressed myocytes but was absent or nearly absent in cells in which one allele of BAG3 was ablated. A), SMAC was present in both the cytoplasm and the mitochondria; however, it was not obvious in the cytoplasm when BAG3 was ablated with an siRNA and TOM22 was also found exclusively in the mitochondria. B) and C), Neither the levels of endonuclease G nor the levels of cIAP1 were altered by any of the consequences of BAG3 biology and its interactions with the cells. SMAC was not seen in the cytoplasm of the cell during periods of stress.

FIGS. 6A-6E show data indicating that levels of proteins known to have altered expression in both animal models of heart failure and in the failing human heart were not alternatively regulated early in the Bag3 deletion model of cardiac failure. A) through C) show the western blots for analysis of the total and phosphorylated forms of JNK, JUN, and ERK1/2. Each blot represents the data for a single study, with an n=5. The data is then presented in a box plot and the p-values are included where appropriate. The data is presented as the phosphorylated form of the protein divided by the total for each protein. As seen in the box plots accompanying each western, there were no statistically significant differences in the levels of these proteins in the tissue obtained from mice with BAG3 haplo-insufficiency when compared to wild-type mice. D) and E), By contrast, western blot analysis revealed that there were significantly higher levels of HuR and C PARP1/GAPDH.

FIGS. 7A-7H show the effects of Bag3 haplo-insufficiency on mitochondrial membrane potential and Ca²⁺ uptake in isolated myocytes and in mitoplasts from Bag3^{+/-} and Bag3^{+/+} mice. LV myocytes were isolated from BAG3^{+/-} and WT mice and exposed to hypoxia for 1 h followed by re-oxygenation for 2 hrs as described in Methods. Cells were permeabilized with digitonin and supplemented with succinate. A) The ratiometric indicator JC-1 was added as indicated by the downward arrow at in order to monitor the membrane potential (ΔΨₘ). The mitochondrial uncoupler CCCP (2 mM) was added at the second arrow. B), A summary of the ΔΨₘ after the addition of Ca²⁺ but before the addition of CCCP (n=4 in each group). C), Extra-mitochondrial Ca²⁺ was measured in a separate group of myocytes after the addition of the ratiometric dye Fura FF at 0 sec. and the subsequent addition of Ca²⁺ pulses (10 mM) as indicated by the (arrows). The cytosolic Ca²⁺ clearance rate was then measured after the first Ca²⁺ pulse as fluorescence arbitrary units. D), A summary of the cytosolic Ca²⁺ clearance rate. n=4 for each measure. *p<0.05, **p<0.01, ***p<0.001. The ΔΨₘ is generated by Ca²⁺ flux through the Ca²⁺-uniporter that is composed of five proteins: MICU1, MICU2, MCUb, MCU and EMRE. E) and F), BAG3 haplo-insufficiency results in a significant (p<0.05) decrease in the relative levels of MICU1 and a trend towards a decrease in MICU2 which leads to an increase (adverse) in membrane function potential. In G) and H), currents from cardiac mitoplasts (I_{MCU}) were recorded before and after application of 5 mM Ca²⁺ to the bath. G), Currents were measured during a voltage-ramp as indicated. Traces are representative single I_{MCU} recordings from WT-GFP, BAG3^{+/-}-GFP and BAG3^{+/-}-BAG3 mitoplasts. H), Means±SEM of I_{MCU} (pA/pF) from WT-GFP (n=5), BAG3^{+/-}-GFP (n=4) and BAG3^{+/-}-BAG3 (n=5) mitoplasts. *p<0.05).

FIGS. 8A-8F show human Bag3 proteome: Bag3 and Bag3-associated protein levels in the failing and non-failing human heart. Tissue was obtained from the left ventricular free wall of human hearts with non-ischemic dilated cardiomyopathy at the time of heart transplantation (IDC) and compared with tissue obtained from non-failing control hearts from transplant donors whose hearts could not be used for transplantation (NF). A-F represent Western blots for each of the indicated proteins with the data summarized in the cumulative figure to the right of each blot. Each study was repeated at least once with comparable results. *p<0.05; **p<0.01.

FIG. 9 is an illustration of the mitochondrial and extra-mitochondrial pathways that are responsible for maintaining mitochondrial homeostasis by regulating the activity of the extrinsic and intrinsic pathways of apoptosis, mitochondrial function and the role of BAG3 in those pathways. Individual proteins include: Bcl2, Bid, tBid, BAX, BAK—members of the Bcl-2 family of proteins that function in both inhibition and stimulation of apoptosis; cIAP-1—the cellular inhibitor of apoptosis-1 (cIAP-2 is formed by the identical gene); —members of the OMM—outer mitochondrial membrane; IMM—inner mitochondrial membrane; MCU—the mitochondrial uniporter; SMAC—the second mitochondrial-derived activator of caspases that is expressed by the DIABLO gene, and promotes apoptosis by activating caspases by blocking the inhibition of caspase activation by cIAP; TNFR1—tumor necrosis factor-alpha receptor; VDAC—voltage-dependent anion channel that is the gatekeeper for the passage of metabolites, nucleotides and ions that play a role in regulating apoptosis by interacting with members of the Bcl-2 family of proteins and hexokinase. Although not wishing to be bound by any theory, when cIAP is attached to a caspase as well as to BAG3-BAG3 stabilizes the cIAP-caspase dimer which in turn prevents SMAC from activating (cleaving) the caspase. When BAG3 binds to the ciIAP that is coupled with the TNFR receptor, it is hypothesized that it stabilizes the receptor complex and doesn't allow TNFalpha to either bind to the receptor or it down-regulates the receptor in such a way that there isn't normal activation of the receptor with subsequent activation of caspase 8.

### DETAILED DESCRIPTION

The following description of certain embodiments is merely exemplary in nature and is in no way intended to limit the invention, its application or uses. Embodiments of the invention may be practiced without the theoretical aspects presented. Moreover, any theoretical aspects are presented with the understanding that Applicant does not seek to be bound by the theory presented.

In certain embodiments, a method of or a formulation for treating a patient suffering from, or, at risk of developing inflammation comprises administering to the patient a therapeutically effective amount of an agent wherein the agent modulates expression or amount of BCL2-associated athanogene 3 (BAG3) encoding nucleic acid, BAG3 protein or BAG3 peptide thereby treating inflammation.

Inflammation includes without limitation, aberrant or undesirable inflammatory responses, autoimmune responses, disorders and diseases. Such responses, disorders and diseases may be antibody or cell mediated, or a combination of antibody and cell mediated. Such responses include T cell or B cell responses.

An inflammatory response refers to any immune response, activity or function that is greater than desired or greater than physiologically normal response, activity or function including, acute or chronic responses, activities or functions. Such inflammatory responses are generally characterized as an undesirable or aberrant increased or inappropriate response, activity or function of the immune system. However, an undesirable inflammatory response, function or activity can be a normal response, function or activity. Thus, normal inflammation or an inflammatory response so long as it is considered undesirable, even if not considered aberrant, is included within the meaning of these terms. An abnormal (aberrant) inflammatory response, function or activity deviates from normal.

Inflammation and inflammatory responses are characterized by many different physiological adverse symptoms or complications, which can be humoral, cell-mediated or a combination thereof. Inflammation, inflammatory responses, disorders and diseases that can be treated in accordance with embodiments herein include, but are not limited to, those that either directly or indirectly lead to or cause cell or tissue/organ damage in a patient. At the whole body, regional or local level, inflammation or inflammatory response can be characterized by swelling, pain, headache, fever, nausea, skeletal joint stiffness, fluid accumulation, lack of mobility, rash, redness or other discoloration. At the cellular level, inflammation can be characterized by one or more of T cell activation and/or differentiation, cell infiltration of the region, production of antibodies, production of cytokines, lymphokines, chemokines, interferons and interleukins, cell growth and maturation factors (e.g., proliferation and differentiation factors), cell accumulation or migration and cell, tissue or organ damage. Thus, methods, uses and formulations include treatment of and an ameliorative effect upon any such physiological symptoms or cellular or biological responses characteristic of inflammation or an inflammatory response.

In particular embodiments, a method, use or formulation according to embodiments herein decreases, reduces, inhibits, suppresses, limits or controls inflammation or an inflammatory response in a patient. In additional particular embodiments, a method, use or formulation decreases, reduces, inhibits, suppresses, limits or controls an adverse symptom of inflammation or an inflammatory response.

Bcl-2 associated anthanogene-3 (BAG3), also known as BCL2-Associated Athanogene 3; MFM6; Bcl-2-Binding Protein Bis;CAIR-1; Docking Protein CAIR-1; BAG Family Molecular Chaperone Regulator 3; BAG-3; BCL2-Binding Athanogene 3; or BIS, is a cytoprotective polypeptide that competes with Hip-1 for binding to HSP 70. The NCBI reference amino acid sequence for BAG3 can be found at Genbank under accession number NP_004272.2; Public GI:14043024. The amino acid sequence of Genbank accession number NP_004272.2; Public GI:14043024 is referred to herein as SEQ ID NO: 1. The NCBI reference nucleic acid sequence for BAG3 can be found at Genbank under accession number NM_004281.3 GI:62530382. The nucleic acid sequence of Genbank accession number NM_004281.3 GI:62530382 is referred as SEQ ID NO: 2. Other BAG3 amino acid sequences include, for example, without limitation, 095817.3 GI:12643665 (SEQ ID NO: 3); EAW49383.1 GI:119569768 (SEQ ID NO: 4); EAW49382.1 GI:119569767(SEQ ID NO: 5); and CAE55998.1 GI:38502170 (SEQ ID NO: 6). The BAG3 polypeptide of the invention can be a can be a variant of a polypeptide described herein, provided it retains functionality.

As used herein, the term “agent” is meant to encompass any molecule, chemical entity, composition, drug, therapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. An agent can be assayed in accordance with the methods of the invention at any stage during clinical trials, during pre-trial testing, or following FDA-approval.

The terms “polypeptides,” “proteins” and “peptides” are used interchangeably herein. The “polypeptides,” “proteins” and “peptides” encoded by the “polynucleotide sequences,” include full-length native sequences, as with naturally occurring proteins, as well as functional subsequences, modified forms or sequence variants so long as the subsequence, modified form or variant retains some degree of functionality of the native full-length protein. Such polypeptides, proteins and peptides encoded by the polynucleotide sequences can be but are not required to be identical to an endogenous protein in the treated patient.

The terms “nucleic acid” and “polynucleotide” are used interchangeably herein to refer to all forms of nucleic acid, oligonucleotides, including deoxyribonucleic acid (DNA) and ribonucleic acid (RNA). Nucleic acids include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA).

Nucleic acids include naturally occurring, synthetic, and intentionally modified or altered polynucleotides. Nucleic acids can be single, double, or triplex, linear or circular, and can be of any length. In discussing nucleic acids, a sequence or structure of a particular polynucleotide may be described herein according to the convention of providing the sequence in the 5′ to 3′ direction.

A “heterologous” polynucleotide or nucleic acid sequence refers to a polynucleotide inserted into a plasmid or vector for purposes of vector mediated transfer/delivery of the polynucleotide into a cell. Heterologous nucleic acid sequences are distinct from viral nucleic acid, i.e., are non-native with respect to viral nucleic acid. Once transferred/delivered into the cell, a heterologous nucleic acid sequence, contained within the vector, can be expressed (e.g., transcribed, and translated if appropriate). Alternatively, a transferred/delivered heterologous polynucleotide in a cell, contained within the vector, need not be expressed. Although the term “heterologous” is not always used herein in reference to nucleic acid sequences and polynucleotides, reference to a nucleic acid sequence or polynucleotide even in the absence of the modifier “heterologous” is intended to include heterologous nucleic acid sequences and polynucleotides in spite of the omission.

The term “expression vector” as used herein refers to a vector containing a nucleic acid sequence (e.g., BAG3) coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

A “promoter” as used herein can refer to a DNA sequence that is typically located adjacent to a nucleic acid sequence (e.g., BAG3). A promoter typically increases an amount of nucleic acid sequence (e.g., BAG3) expressed compared to an amount expressed when no promoter exists.

An “enhancer” as used herein can refer to a sequence that is located adjacent to the nucleic acid sequence (e.g., BAG3). Enhancer elements are typically located upstream of a promoter element but also function and can be located downstream of or within a nucleic acid sequence (e.g., BAG3). Hence, an enhancer element can be located 100 base pairs, 200 base pairs, or 300 or more base pairs upstream or downstream of a nucleic acid sequence (e.g., BAG3). Enhancer elements typically increase expression of a nucleic acid sequence (e.g., BAG3) above increased expression afforded by a promoter element.

Examples of expression regulatory elements or expression control elements that can be used in methods according to the invention, include, for example and without limitation, cytomegalovirus (CMV) immediate early promoter/enhancer, Rous sarcoma virus (RSV) promoter/enhancer, SV40 promoter, dihydrofolate reductase (DHFR) promoter, chicken β-actin (CBA) promoter, phosphoglycerol kinase (PGK) promoter, and elongation factor-1 alpha (EF1-alpha) promoter.

In certain embodiments, viral vectors that may be used in the invention methods and formulations include, for example and without limitation, AAV particles. In certain embodiments, viral vectors that may be used in the invention include, for example and without limitation, retroviral, adenoviral, helper-dependent adenoviral, hybrid adenoviral, herpes simplex virus, lentiviral, poxvirus, Epstein-Barr virus, vaccinia virus, and human cytomegalovirus vectors, including recombinant versions thereof.

The term “recombinant,” as a modifier of a viral vector, such as a recombinant AAV (rAAV) vector, as well as a modifier of sequences such as recombinant polynucleotides and polypeptides, means that compositions have been manipulated (i.e., engineered) in a fashion that generally does not occur in nature. A “recombinant viral vector” therefore refers to a viral vector comprising one or more heterologous gene products or sequences.

Since many viral vectors exhibit size-constraints associated with packaging, the heterologous gene products or sequences are typically introduced by replacing one or more portions of the viral genome. Such viruses may become replication-defective, requiring the deleted function(s) to be provided in trans (i.e., “helper” function) during viral replication and encapsidation (by using, e.g., a helper virus or a packaging cell line carrying gene products necessary for replication and/or encapsidation, such as AAV rep, AAV cap, human adenoviral E4 and adenoviral VA RNA). Modified viral vectors in which a polynucleotide to be delivered is carried on the outside of the viral particle have also been described (see, e.g., Curiel, D T, et al., PNAS 88:8850-8854, 1991).

A particular example of a recombinant AAV vector would be where a nucleic acid that is not normally present in a wild-type AAV genome (heterologous polynucleotide) is inserted within a viral genome. An example of which would be where a nucleic acid (e.g., gene) encoding a therapeutic protein or polynucleotide sequence is cloned into a vector, with or without 5′, 3′ and/or intron regions that the gene is normally associated within the AAV genome. Although the term “recombinant” is not always used herein in reference to an AAV vector, as well as sequences such as polynucleotides, recombinant forms including AAV vectors, polynucleotides, etc., are expressly included in spite of any such omission.

A “rAAV vector,” for example, is derived from a wild-type genome of AAV by using molecular methods to remove all or a part of a wild-type AAV genome, and replacing with a non-native (heterologous) nucleic acid, such as a nucleic acid encoding a therapeutic protein or polynucleotide sequence. Typically, for a rAAV vector one or both inverted terminal repeat (ITR) sequences of AAV genome are retained. A rAAV is distinguished from an AAV genome since all or a part of an AAV genome has been replaced with a non-native sequence with respect to the AAV genomic nucleic acid, such as with a heterologous nucleic acid encoding a therapeutic protein or polynucleotide sequence. Incorporation of a non-native (heterologous) sequence therefore defines an AAV as a “recombinant” AAV vector, which can be referred to as a “rAAV vector.”

A recombinant AAV vector sequence (or genome) can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV,” “rAAV particle” and/or “rAAV virion.” Such rAAV, rAAV particles and rAAV virions include proteins that encapsidate or package a vector genome. Particular examples include in the case of AAV, capsid proteins.

A “vector genome,” which may be abbreviated as “vg,” refers to the portion of the recombinant plasmid sequence that is ultimately packaged or encapsidated to form a rAAV particle. In cases where recombinant plasmids are used to construct or manufacture recombinant AAV vectors, the AAV vector genome does not include the portion of the “plasmid” that does not correspond to the vector genome sequence of the recombinant plasmid. This non-vector genome portion of the recombinant plasmid is referred to as the “plasmid backbone,” which is important for cloning and amplification of the plasmid, a process that is needed for propagation and recombinant AAV vector production, but is not itself packaged or encapsidated into rAAV particles. Thus, a “vector genome” refers to the nucleic acid that is packaged or encapsidated by rAAV.

As used herein, the term “serotype” in reference to an AAV vector means a capsid that is serologically distinct from other AAV serotypes. Serologic distinctiveness is determined on the basis of lack of cross-reactivity between antibodies to one AAV as compared to another AAV. Cross-reactivity differences are usually due to differences in capsid protein sequences/antigenic determinants (e.g., due to VP1, VP2, and/or VP3 sequence differences of AAV serotypes). An antibody to one AAV may cross-react with one or more other AAV serotypes due to homology of capsid protein sequence.

Under the traditional definition, a serotype means that the virus of interest has been tested against serum specific for all existing and characterized serotypes for neutralizing activity and no antibodies have been found that neutralize the virus of interest. As more naturally occurring virus isolates are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

rAAV vectors include any viral strain or serotype. For example and without limitation, a rAAV vector genome or particle (capsid, such as VP1, VP2 and/or VP3) can be based upon any AAV serotype, such as AAV-1, -2, -3, -4, -5, -6, -7, -8, -9, -10, -11, -12, AAV3B or AAV-2i8, for example. Such vectors can be based on the same strain or serotype (or subgroup or variant), or be different from each other. For example and without limitation, a rAAV plasmid or vector genome or particle (capsid) based upon one serotype genome can be identical to one or more of the capsid proteins that package the vector. In addition, a rAAV plasmid or vector genome can be based upon an AAV serotype genome distinct from one or more of the capsid proteins that package the vector genome, in which case at least one of the three capsid proteins could be a different AAV serotype, e.g., AAV1, AAV2, AAV3, AAV3B, AAV-2i8 (AAV2/AAV8 chimera), AAV4, AAV5, AAV6, AAV7, AAV8, AAV9, AAV10, AAV11, AAV12, or variant thereof, for example. More specifically, a rAAV2 vector genome can comprise AAV2 ITRs but capsids from a different serotype, such as AAV1, AAV3, AAV3B, AAV4, AAV5, AAV6, AAV7, AAV8, AAV9, AAV10, AAV11, AAV12, AAV-2i8, or variant thereof, for example. Accordingly, rAAV vectors include gene/protein sequences identical to gene/protein sequences characteristic for a particular serotype, as well as “mixed” serotypes, which also can be referred to as “pseudotypes.”

In certain embodiments, a rAAV vector includes or consists of a capsid sequence at least 70% or more (e.g., 75%, 80%, 85%, 90%, 95%, 96%, 97%, 98%, 99%, 99.5%, etc.) identical to one or more AAV1, AAV2, AAV3, AAV3B, AAV-2i8, AAV4, AAV5, AAV6, AAV7, AAV8, AAV9, AAV10, AAV11, or AAV12 capsid proteins (VP1, VP2, and/or VP3 sequences). In certain embodiments, a rAAV vector includes or consists of a sequence at least 70% or more (e.g., 75%, 80%, 85%, 90%, 95%, 96%, 97%, 98%, 99%, 99.5%, etc.) identical to one or more AAV1, AAV2, AAV3, AAV3B, AAV4, AAV5, AAV6, AAV7, AAV8, AAV9, AAV10, AAV11, or AAV12 ITR(s).

In certain embodiments, rAAV vectors include AAV1, AAV2, AAV3, AAV3B, AAV-2i8, AAV4, AAV5, AAV6, AAV7, AAV8, AAV9, AAV10, AAV11, or AAV12 variants (e.g., ITR and capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. application Ser. No. 13/594,773).

rAAV, such as AAV1, AAV2, AAV3, AAV3B, AAV-2i8, AAV4, AAV5, AAV6, AAV7, AAV8, AAV9, AAV10, AAV11, AAV12 and variants, hybrids and chimeric sequences, can be constructed using recombinant techniques that are known to a skilled artisan, to include one or more heterologous polynucleotide sequences (transgenes) flanked with one or more functional AAV ITR sequences. Such AAV vectors typically retain at least one functional flanking ITR sequence(s), as necessary for the rescue, replication, and packaging of the recombinant vector into a rAAV vector particle. A rAAV vector genome would therefore include sequences required in cis for replication and packaging (e.g., functional ITR sequences).

In certain embodiments, a lentivirus used in the invention may be a human immunodeficiency-1 (HIV-1), human immunodeficiency-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), or caprine arthritis encephalitis virus (CAEV). Lentiviral vectors are capable of providing efficient delivery, integration and long-term expression of heterologous polynucleotide sequences into non-dividing cells both in vitro and in vivo. A variety of lentiviral vectors are known in the art, see Naldini et al. (Proc. Natl. Acad. Sci. USA, 93:11382-11388 (1996); Science, 272: 263-267 (1996)), Zufferey et al., (Nat. Biotechnol., 15:871-875, 1997), Dull et al., (J Virol. 1998 November; 72(11):8463-71, 1998), U.S. Pat. Nos. 6,013,516 and 5,994,136, any of which may be a suitable viral vector for use in the invention.

Recombinant viral vector doses can be formulated, administered or delivered at any appropriate dose. Generally, doses will range from at least 1×10⁸, or more, for example, 1×10⁹, 1×10¹⁰, 1×10¹¹, 1×10¹², 1×10¹³ or 1×10¹⁴, or more, vector genomes per kilogram (vg/kg) of the weight of the patient, to achieve an effect. AAV dose in the range of 1×10¹⁰-1×10¹¹ vg/kg in mice, and 1×10¹²-1×10¹³ vg/kg in dogs have been effective. More particularly, a dose from about 1×10¹¹ vg/kg to about 5×10¹⁴ vg/kg inclusive, or from about 5×10¹¹ vg/kg to about 1×10¹⁴ vg/kg inclusive, or from about 5×10¹¹ vg/kg to about 5×10¹³ vg/kg inclusive, or from about 5×10¹¹ vg/kg to about 1×10¹³ vg/kg inclusive, or from about 5×10¹¹ vg/kg or about 5×10¹² vg/kg inclusive, or from about 5×10¹¹ vg/kg to about 1×10¹² vg/kg inclusive. Doses can be, for example, about 5×10¹⁴ vg/kg, or less than about 5×10¹⁴ vg/kg, such as a dose from about 2×10¹¹ to about 2×10¹⁴ vg/kg inclusive, in particular, for example, about 2×10¹² vg/kg, about 6×10¹² vg/kg, or about 2×10¹³ vg/kg.

An “effective amount,” “sufficient amount” or “therapeutically effective amount” refers to an amount that provides, in single or multiple doses, alone or in combination, with one or more other compositions, treatments, protocols, or therapeutic regimens agents, a detectable response of any duration of time (long or short term), an expected or desired outcome in or a benefit to a patient of any measurable or detectable degree or for any duration of time (e.g., for minutes, hours, days, months, years, or cured). The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single formulation or administration, may require multiple administrations, and can but need not be, administered alone or in combination with another composition (e.g., agent), treatment, protocol or therapeutic regimen. For example, the amount may be proportionally increased as indicated by the need of the patient, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given patient. Amounts considered effective also include amounts that result in a reduction of the use of another treatment, therapeutic regimen or protocol.

An effective amount or a sufficient amount need not be effective in each and every patient treated, nor a majority of treated patients in a given group or population. An effective amount or a sufficient amount means effectiveness or sufficiency in a particular patient, not a group or the general population. As is typical for such methods, some patients will exhibit a greater response, or less or no response to a given treatment method or use.

Methods, uses and formulations of the invention therefore include providing a detectable or measurable beneficial effect to a patient, or any objective or subjective transient or temporary, or longer-term improvement (e.g., cure) in the inflammation, or inflammatory response. Thus, a satisfactory clinical endpoint is achieved when there is an incremental improvement in the patient's condition or a partial reduction in severity, frequency, duration or progression of one or more associated adverse symptoms or complications of inflammation or an inflammatory response, or inhibition, reduction, elimination, prevention or reversal of one or more of the physiological, biochemical or cellular manifestations or characteristics of inflammation or an inflammatory response. A therapeutic benefit or improvement (“ameliorate” is used synonymously) therefore need not be complete ablation of any or all adverse symptoms or complications associated with inflammation or an inflammatory response but is any measurable or detectable, objectively or subjectively, meaningful improvement in the inflammation or inflammatory response. For example, inhibiting a worsening or progression of inflammation or an inflammatory response, or an associated symptom (e.g., slowing progression or stabilizing one or more symptoms, complications or physiological or psychological effects or responses), even if only for a few days, weeks or months, even if complete ablation of the inflammation or inflammatory response, or an associated adverse symptom is not achieved is considered to be a beneficial effect.

“Treatment” is an intervention performed with the intention of preventing the development, altering the pathology or symptoms of a disorder or delaying progression or worsening of a disorder. Accordingly, “treatment” refers to both therapeutic treatment and prophylactic or preventative measures. “Treatment” may also be specified as palliative care.

“Prophylaxis” and grammatical variations thereof mean a method in accordance with the invention in which contact, administration or in vivo delivery to a subject is prior to manifestation or onset of a condition, disorder or disease (or an associated symptom or physiological or psychological response), such that it can eliminate, prevent, inhibit, decrease or reduce the probability, susceptibility, onset or frequency of having a condition, disorder or disease, or an associated symptom. Target patients for prophylaxis can be one of increased risk (probability or susceptibility) of contracting inflammation or inflammatory response, or an associated symptom, or recurrence of a previously diagnosed inflammation or inflammatory response, or an associated symptom, as set forth herein.

Those in need of treatment include those already with the disorder as well as those in which the disorder is to be prevented. Accordingly, “treating” or “treatment” of a disorder or condition includes; (1) preventing or delaying the appearance of clinical symptoms of the state, disorder or condition developing in a human or other mammal that may be afflicted with or predisposed to the disorder or condition but does not yet experience or display clinical or subclinical symptoms of the disorder or condition; (2) inhibiting the disorder or condition, i.e., arresting, reducing or delaying the development of the disorder or condition or a relapse thereof (in case of maintenance treatment) or at least one clinical or subclinical symptom thereof; or (3) relieving the disease, i.e., causing regression of the disorder or condition or at least one of its clinical or subclinical symptoms. The benefit to a patient to be treated is either statistically significant or at least perceptible to the patient or to the physician.

The term “ameliorate” means a detectable or measurable improvement in a patient's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease.

Formulations can be administered one from one or more times per day; once every other day; one or more times per week; one or more times per month; one or more times per year; or 1-2 times over the patient's lifetime. The skilled artisan will appreciate that certain factors can influence the dosage and timing required to treat a patient, including but not limited to the severity of the disease or disorder, desired outcome, previous treatments, the general health and/or age of the patient, and other diseases present. Moreover, treatment of a patient with a therapeutically effective amount in accordance with the invention can include a single treatment or multiple treatments, such as a series of treatments.

Formulations, compositions and pharmaceutical compositions of the invention include compositions wherein the active agent is contained in an effective amount to achieve the intended therapeutic purpose. Determining an effective dose is well within the capability of a skilled medical practitioner using techniques and guidance known in the art and using the teachings provided herein.

Formulations, such as pharmaceutical compositions, may be delivered to a patient, so as to allow nucleic acid transcription and translation of encoded protein. In certain embodiments, formulations, such as pharmaceutical compositions, comprise sufficient genetic material to enable production of a therapeutically effective amount of BAG3 in the patient, for example, to modulate TNF signaling.

By the term “modulate,” it is meant that any of the mentioned activities of the compounds embodied herein, are, e.g., increased, enhanced, increased, agonized (acts as an agonist), promoted, decreased, reduced, inhibited, suppressed, blocked or antagonized (acts as an antagonist). Modulate can reduce or decrease its activity below baseline values, e.g., a reduction or decrease of 1 to 5 fold, 1 to 10 fold, 5 to 10 fold, 10 to 20 fold, 20 to 30 fold, 40 to 50 fold, etc., or at least 1-fold, 2-fold, 3-fold, 5-fold, 10-fold, 20 fold, 50 fold 100-fold, etc. Modulate also can increase or enhance activity over baseline values, e.g., an increase or enhancement of 1 to 5 fold, 1 to 10 fold, 5 to 10 fold, 10 to 20 fold, 20 to 30 fold, 40 to 50 fold, etc., or at least 1-fold, 2-fold, 3-fold, 5-fold, 10-fold, 20 fold, 50 fold 100-fold, etc.

Invention formulations, compositions, methods and uses can be used in primate (e.g., human) and veterinary medical applications. Suitable patients therefore include mammals, such as humans, as well as non-human mammals. The terms “patient” and “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human patients include fetal, neonatal, infant, juvenile and adult subjects. Patients also include animal disease models, for example, mouse and other animal models of BAG3 insufficiency.

Compositions and formulations may be sterile and methods and uses may be practiced with sterile compositions and formulations. Compositions may be formulated with or be administered in any biocompatible pharmaceutical carrier, including, but not limited to, saline, buffered saline, dextrose, and water. The compositions may be formulated or administered to a patient alone, or in combination with other agents, which influence dosage amount, administration frequency and/or therapeutic efficacy.

Formulations, methods and uses of the invention include delivery and administration systemically, regionally or locally (e.g., to a particular region, tissue, organ or cell), or by any route, for example, by injection or infusion. Administration or delivery of the compositions, formulations and pharmaceutical compositions in vivo may generally be accomplished via injection using a conventional syringe, although other delivery methods such as convection-enhanced delivery are envisioned (See e.g., U.S. Pat. No. 5,720,720). For example, formulations and compositions may be delivered subcutaneously, epidermally, intradermally, intrathecally, intraorbitally, intramucosally, intraperitoneally, intravenously, intra-pleurally, intraarterially, orally, intrahepatically or intramuscularly. A clinician specializing in the treatment of patients may determine the optimal route for administration based on a number of criteria, including, but not limited to, the condition of the patient and the purpose of the treatment (e.g., modulating TNF signaling, reducing TNF signaling, treating inflammation, reducing an inflammatory response, etc.).

Also in accordance with the invention, nucleic acids, expression vectors including viral vectors and viral particles may be encapsulated or complexed with liposomes, nanoparticles, lipid nanoparticles, polymers, microparticles, microcapsules, micelles, or extracellular vesicles.

A “lipid nanoparticle” or “LNP” refers to a lipid-based vesicle useful for administration or delivery of nucleic acids, expression vectors including viral vectors having dimensions on the nanoscale, i.e., from about 10 nm to about 1000 nm, or from about 50 to about 500 nm, or from about 75 to about 127 nm. Without being bound by theory, LNP is believed to provide nucleic acid, expression vector or recombinant viral vector with partial or complete shielding from the immune system. Shielding allows delivery of the nucleic acid, expression vector or viral vector to a tissue or cell while avoiding inducing a substantial immune response against the nucleic acid, expression vector or viral vector in vivo. Shielding may also allow repeated administration without inducing a substantial immune response. Shielding may also improve or increase delivery efficiency, duration of therapeutic effect and/or therapeutic efficacy in vivo.

The AAV surface carries a slight negative charge. As such it may be beneficial for the LNP to comprise a cationic lipid such as, for example, an amino lipid. Exemplary amino lipids have been described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411 and 2010/0117125.

The terms “cationic lipid” and “amino lipid” are used interchangeably herein to include those lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipids may also be titratable cationic lipids. In certain embodiments, the cationic lipids comprise: a protonatable tertiary amine (e.g., pH-titratable) group; C18 alkyl chains, wherein each alkyl chain independently has 0 to 3 (e.g., 0, 1, 2, or 3) double bonds; and ether, ester, or ketal linkages between the head group and alkyl chains.

In certain embodiments, cationic lipid may be present in an amount from about 10% by weight of the LNP to about 85% by weight of the lipid nanoparticle, or from about 50% by weight of the LNP to about 75% by weight of the LNP.

LNP can comprise a neutral lipid. Neutral lipids may comprise any lipid species which exists either in an uncharged or neutral zwitterionic form at physiological pH. Such lipids include, without limitation, diacylphosphatidylcholine, diacylphosphatidylethanolamine, ceramide, sphingomyelin, dihydrosphingomyelin, cephalin, and cerebrosides. The selection of neutral lipids is generally guided by consideration of, inter alia, particle size and the requisite stability. In certain embodiments, the neutral lipid component may be a lipid having two acyl groups (e.g., diacylphosphatidylcholine and diacylphosphatidylethanolamine).

In certain embodiments, the neutral lipid may be present in an amount from about 0.1% by weight of the lipid nanoparticle to about 75% by weight of the LNP, or from about 5% by weight of the LNP to about 15% by weight of the LNP.

A biological sample is typically obtained from or produced by a biological organism. Examples of biological samples from a patient that may be analyzed include, for example and without limitation, whole blood, serum, plasma, the like, and a combination thereof. Other biological samples from a patient include, for example and without limitation, cerebrospinal fluid or simply spinal fluid. A biological sample may be devoid of cells, or may include cells (e.g., red blood cells, platelets and/or lymphocytes).

The invention provides compositions, such as kits, that include packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, virus (e.g., AAV, lentivirus) vector, or virus particle.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or patient for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the patient or clinician regarding situations where it would not be appropriate to use a particular composition. Adverse side effects or complications could also occur when the patient has, will be or is currently taking one or more other medications that may be incompatible with the composition, or the patient has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component.

Unless otherwise defined, all technical and scientific terms used herein have the same meaning as commonly understood by one of ordinary skill in the art to which the invention belongs. Although methods and materials similar or equivalent to those described herein can be used in the practice or testing of the invention, suitable methods and materials are described herein.

All patents, patent applications, publications, and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, the terms “comprising,” “comprise” or “comprised,” and variations thereof, in reference to defined or described elements of an item, composition, formulation, method, process, system, etc. are meant to be inclusive or open ended, permitting additional elements, thereby indicating that the defined or described item, composition, formulation, method, process, system, etc. includes those specified elements—or, as appropriate, equivalents thereof—and that other elements can be included and still fall within the scope/definition of the defined item, composition, formulation, method, process, system, etc.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, or up to 10%, or up to 5% within a given value. Alternatively, particularly with respect to biological systems or processes, the term can mean within an order of magnitude, for example within 5-fold, 4-fold, 3-fold, or within 2-fold, of a given value. Where particular values are described in the application and claims, unless otherwise stated the term “about” meaning within an acceptable error range for the particular value should be assumed.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100% etc., as well as 95.1%, 95.2%, 95.3%, 95.4%, 95.5%, etc., 96.1%, 96.2%, 96.3%, 96.4%, 96.5%, etc., and so forth. Thus, to also illustrate, reference to a numerical range, such as “1-4” includes 2, 3, as well as 1.1, 1.2, 1.3, 1.4, etc., and so forth. For example, “1 to 4 weeks” includes 7, 8, 9, 10, 11, 12, 13, 14, 15, 16, 17, 18, 19, 20, 21, 22, 23, 24, 25, 26, 27, or 28 days.

Further, reference to a numerical range, such as “0.01 to 10” includes 0.011, 0.012, 0.013, etc., as well as 9.5, 9.6, 9.7, 9.8, 9.9, etc., and so forth. For example, a dosage of about “0.01 mg/kg to about 10 mg/kg” body weight of a patient includes 0.011 mg/kg, 0.012 mg/kg, 0.013 mg/kg, 0.014 mg/kg, 0.015 mg/kg etc., as well as 9.5 mg/kg, 9.6 mg/kg, 9.7 mg/kg, 9.8 mg/kg, 9.9 mg/kg etc., and so forth.

Reference to an integer with more (greater) or less than includes any number greater or less than the reference number, respectively. Thus, for example, reference to more than 2 includes 3, 4, 5, 6, 7, 8, 9, 10, 11, 12, 13, 14, 15, etc., and so forth. For example, administration of a recombinant viral vector, protease and/or glycosidase “two or more” times includes 3, 4, 5, 6, 7, 8, 9, 10, 11, 12, 13, 14, 15, or more times.

Further, reference to a numerical range, such as “1 to 90” includes 1.1, 1.2, 1.3, 1.4, 1.5, etc., as well as 81, 82, 83, 84, 85, etc., and so forth. For example, “between about 1 minute to about 90 days” includes 1.1 minutes, 1.2 minutes, 1.3 minutes, 1.4 minutes, 1.5 minutes, etc., as well as one day, 2 days, 3 days, 4 days, 5 days . . . 81 days, 82 days, 83 days, 84 days, 85 days, etc., and so forth.

“Optional” or “optionally” means that the subsequently described circumstance may or may not occur, such that the description includes instances where the circumstance occurs and instances where it does not.

The invention is generally disclosed herein using affirmative language to describe the numerous embodiments of the invention. The invention also specifically includes embodiments in which particular subject matter is excluded, in full or in part, such as compositions or formulations, uses, method steps and conditions, protocols, or procedures. For example, in certain embodiments of the invention, compositions and/or method steps are excluded. Thus, even though the invention is generally not expressed herein in terms of what the invention does not include, aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### EXAMPLES

### Example 1: Materials and Methods

Animals, animal models, surgical procedures and human tissue Mice in which a single allele or two alleles of BAG3 were deleted were generated by crossing mice carrying a floxed BAG3 (BAG3^{flox/flox}) with Cre mice carrying α-myosin heavy chain (α-MHC) (all on a C57Bl/6 background) as previously described (10). BAG3^{flox/flox} mice (BAG3 (HEPD0556_7_B06) were obtained from MRC Harwell (a member of the International Mouse Phenotyping Consortium) that generates and distributes transgenic mice on behalf of the European Mouse Mutant Archive (www.infrafrontier.eu). The phenotypes of the Bag3^{+/-} and Bag3^{-/-} mice were described previously,(10) Samples of failing and non-failing human heart were obtained from the heart tissue repositories at the University of Pittsburgh and the University of Colorado Health Sciences Campus as described previously (23,24).

Mass Spectromety—Bag3^{+/+} (WT) and Bag3^{+/-} Mice

Left ventricular tissue from three wild-type and three Bag3-KO mice were homogenized in a lysis buffer containing 9M urea and then briefly sonicated. The solution was then centrifuged at 10,000 RCF for 10 minutes and the supernatant containing the solubilized protein was collected. Protein concentration was determined by BCA assay (Pierce). Approximately 300 μg of protein from each sample were used for proteomics.

The raw mass spectrometry data were imported into the Peaks Bioinformatics software and searched against the Mus musculus database with carbamidomethylated cysteine as the fixed modification and phosphorylation as the variable modification. The data were analyzed using the built-in label free quantification (LFQ) option normalized to the total ion current (TIC) of each sample. Proteins of interest identified by this method were further examined by pathway analysis using the DAVID bioinformatics program (Version 6.8), which allowed for protein function and cell compartment characterization.

### TUNEL Staining for Cell Death

Isolated adult mouse cardiomyocytes were plated on 35 mm² dishes with a No. 1.5, 10 mm² glass diameter coverslip insert (MatTek Corporation Cat #P35G-1.5-10-C) coated with laminin. Cells were subjected to hypoxia (1 hr) and reoxygenation (2 hr) before staining with 100 nM Nonyl Acridine Orange (Molecular Probes Cat #A1372) in Tyrode's buffer. Cells were then imaged by confocal microscopy or fluorescent microscopy as previously described.

### TMRM Stain for Mitochondrial Membrane Potential and Mitochondria Content

Isolated adult mouse cardiomyocytes were plated as described in the TUNEL staining section. Cells were stained with 100 nM Tetramethylrhodamine, Methyl Ester, Perchlorate (TMRM) (Thermo Fisher Scientific Cat #T668) in Tyrode's buffer. Imaging was performed as described previously by confocal microscopy.

### MitoSOX Staining for Mitochondrial ROS Content

Isolated adult mouse cardiomyocytes were plated as described in the TUNEL staining section. Cells were stained with 5 μM MitoSOX™ Red Mitochondrial Superoxide Indicator (Thermo Fisher Scientific Cat #M36008) and confocal images were quantified in Fiji Image J.

### Preparation of Primary Neonatal Mouse Ventricular Cardiomyocytes (NMVCs)

Neonatal mouse ventricular cardiomyocytes were isolated from 1 to 3 day old FVB mice using a Pierce Primary Cardiomyocyte Isolation Kit (Cat no. 88281, Thermo Scientific, Rockford IL) according to manufacturer's instructions as described previously (27).

### Isolation and Culture of Adult Myocytes

Adult cardiac myocytes were isolated form the septum and LV free wall of Bag3^{+/+} (WT), Bag3^{-/-}, and Bag3^{+/-} mice, plated on laminin-coated glass coverslips and then handled as first described by Zhou et al (28) with later published modifications by members of this research team (29),(30). A detailed description of the experimental technique has been described previously.

### Bag3 Knockdown

NMVCs at 60-70% confluence were transfected using the lipofectamine 3000 system (Thermo Scientific, Waltham, MA) with a Bag3 specific siRNA along with lipofectamine RNAimax (ThermoFisher) according to manufacturer's instructions as described previously.

### Hypoxia/Re-Oxygenation (H/R)

NMVCs were subjected to H/R as described previously (31). In brief, NMVCs were exposed to humidified 5% CO₂: 95% N₂ for 16 hours at 37° C. and incubated in glucose free medium. Cells were then re-oxygenated with 5% CO₂:95% humidified air for 4 hours in medium containing glucose.

### Cell Harvest and Protein Extraction

Cultured cells were washed in 1×PBS and lysed in lysis buffer supplemented with mammalian protease inhibitor cocktail and then scraped from the dish. Cells were vortexed and then centrifuged at 13,000×g for 5 min in the cold. The supernatant was collected and used for protein analysis (27).

### Protein Isolation

Hearts were excised, the left ventricles were separated, flash frozen in liquid nitrogen and stored at −80° C. until use. Membrane proteins were prepared as described previously (32) using a Bullet Blender (Next Advance, Averill Park, NY).

### Cytoplasmic and Mitochondrial Fractionation

Mitochondrial and cytoplasmic proteins were separated using a Mitochondria Isolation Kit for Cultured Cells (Thermofisher, #89874) following manufacturer's instructions. Isolated protein was quantified using the Braford assay (Bio-Rad, USA). Proteins were then separated by western blot analysis.

### Immunoprecipitation

NVCMs or AC16 cardiomyocytes were plated onto a 10 cm dish and treated as described above. Cells were quickly washed with cold PBS and placed into IP lysis buffer (Thermofisher) that was supplemented with phosphates inhibitor and halt proteinase inhibitor and homogenized with beads in a bullet blender. The protein lysate was then incubated with Magna magnetic beads (A/G) (Millipore, Sigma) for an hour and the amount of lysate was quantified as described previously.

### Western Blot Analysis

Protein lysate (90 μl) was mixed with a reducing agent (ThermoFisher,) and proteins were separated using a NuPAGE Gel (ThermoFisher) and transferred to nitrocellulose membranes (LiCor, Lincoln, NE) by using a wet transfer system as described previously (17). Membranes were quickly washed, blocked with Licor Odyssey blocking buffer (LiCor) and incubated with the secondary antibody and resulting images were captured with a Licor imaging system.

### Immunofluorescent Staining

Neonatal or adult cardiomyocytes were fixed with 4% paraformaldehyde in PBS for 15 minutes and washed; permeabilized with 0.5% Triton X-100 in PBS for 10 minutes and washed; blocked with Licor blocking buffer S containing 5% bovine serum albumin (BSA) and 0.1% Triton X-100 for 1 hour; all at room temperature. The cells were incubated overnight at 4° C. with rabbit anti-protein of interest antibody diluted in blocking solution. Cells were then washed with PBS and incubated for 45 minutes at room temperature with suitable secondary antibody and DAPI (4′,6-diamidino-2-phenylindole) diluted in blocking solution.

### Confocal MICROSCOPY

Confocal microscopy was used to detect in adult cardiomyocytes as described previously (17). Briefly, NMVCs were isolated and plated on laminin-coated 4-well chamber slides (Lab-Tek., Rochester, NY). Bag3 was identified using a primary rabbit antibody (1:200; Proteintech Group Inc, Chicago IL) (28,29). Total laser intensity and photomultiplier gain were set constant for all groups and settings and data were verified by two independent observers who were blinded to the experimental group. A minimum of three coverslips were used for each experimental group and at least three cell images were acquired from each coverslip.

Measurement of Mitochondrial Membrane Potential (ΔΨₘ) and mitochondrial Ca²⁺ Uptake

Measurements were made as described previously (35). In brief, LV myocytes isolated from WT and Bag3^{+/-} hearts were exposed to either 21% O₂-5% CO₂ (normoxia) or 1% O₂-5% CO₂ (hypoxia) for 30 min followed by 30 min of reoxygenation.(33) Permeabilized myocytes were supplemented with succinate. Fura-FF (0.5 μM) was added at 0 s and JC-1 (800 nM; Molecular Probes) at 20 s to measure extra-mitochondrial Ca²⁺ and ΔΨₘ, respectively. Fluorescence signals were monitored with multiwavelength-excitation and a dual wavelength-emission spectrofluorometer (Delta RAM, Photon Technology International), The ratiometric dye Fura-FF was calibrated as previously described (30). At times indicated, 10 μM Ca²⁺ pulse was added and ΔΨₘ and extra-mitochondrial Ca²⁺ were monitored simultaneously. ΔΨₘ was calculated as the ratio of the fluorescence of the JC-1 oligomeric to monomeric forms. Cytosolic Ca²⁺ clearance rate was taken to represent mitochondrial Ca²⁺ uptake.

### Echocardiography

Global LV function was evaluated in all mice after light sedation (2% isoflurane) using a VisualSonics Vevo 770 imaging system and a 707 scan head (Miami, FL) as described previously.(11) The left ventricular ejection fraction (LVEF) was calculated using the formula EF %=[(LVEDV−LVESV)/LVEDV]×100; where LVEDV and LVESV are left ventricular end-diastolic volume and left ventricular end-systolic volume, respectively.

Measurement of Mitochondrial Ca²⁺ uniporter (MCU) current (I_{MCU})

Myocytes were isolated from LV and septum of 8 to 12 week old WT or BAG3^{+/-} mice.(29) Myocytes were then infected with Adv-GFP or Adv-BAG3 (7×10⁶ pfu/ml) and cultured for 24 before use for mitoplast isolation (34). Mitoplast patch clamp recordings were conducted at 30° C. as previously described in detail (35-37). I_{MCU} was recorded using a computer controlled Axon200B patch-clamp amplifier with a Digidata 1320A acquisition board (pClamp 10.0 software; Axon Instruments). Mitoplasts were bathed in physiologic solutions and after formation of GQ seals mitoplasts were ruptured and capacitance was measured. After capacitance compensation, mitoplasts were held at 0 mV and I_{MCU} was elicited with a voltage ramp (from −160 mV to +80 mV, 120 mV/s) both before and after addition of 5 mM Ca.

### Statistical Analysis

Data are presented as means±SEM for continuous variables. ANOVA with Bonferroni multiple comparisons adjustments were used to assess differences across the investigational groups. For Western blot analysis a p-value of p<0.05 was considered significant. The control for each experiment (eg. Ad-GFP or normoxia) was set as 1.0. The full blots used for assessment of protein levels including the loading standard are shown for each experiment and each experiment was replicated at least once and achieved comparable results. All blots were normalized by the appropriate standard from the same gel as the blot. Individual elements on each figure represent a distinct biological measurement in one sample. No technical replicates are seen in the result sections of this study unless noted. The original blot for each figure is available upon request from verifiable scientists. In the case of measurement of the levels of the multiple components of the uniporter, each experiment was repeated five times because of the small protein yield in each experiment.

### Example 2: Results

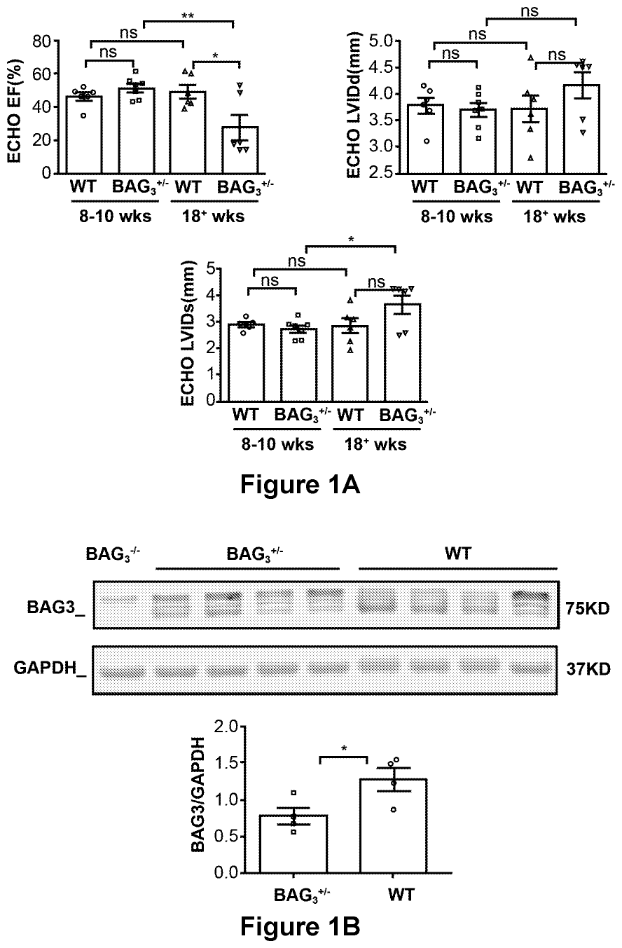

Ablation of one allele of BAG3 in mice results in age-related LV dysfunction Mice with a single allele knockout of Bag3 provide an ideal model in which to study the biology of BAag3 because they mirror the molecular biology of deletions or truncations.(8-10,27,38) As seen in FIG. 1A, 8 to 10 week-old mice in whom one allele of BAG3 was ablated (Bag3^{+/+}) have a normal LV phenotype by echocardiography. However, by 18 weeks of age, Bag3^{+/-} mice have a significant diminution in LV function as well as LV enlargement. Not unexpectedly, levels of Bag3 were consistently diminished by approximately 50% in all Bag3^{+/-} groups (FIG. 1B). Young Bag3^{+/-} mice therefore provided an ideal model in which to study the effects of Bag3 deficiency on the cell and molecular biology of the heart.

Cardiomyocyte-Restricted Bag3 KO in Mice is Associated with Altered Mitochondrial Protein Expression.

An approximately 50% reduction in Bag3 expression in the heart is associated with heart failure in humans (11,12) and cardiomyocyte-restricted Bag3 haploinsufficiency in mice causes progressive left ventricular dysfunction consistent with the phenotype in humans (10). In order to characterize the impact of decreased Bag3 in the heart and gain insight into which specific pathways are dysregulated, unbiased mass spectrometry was used to analyze the proteome of young mice with cardiomyocyte-specific Bag3 knockout compared to age-matched wild-type controls (9,10).

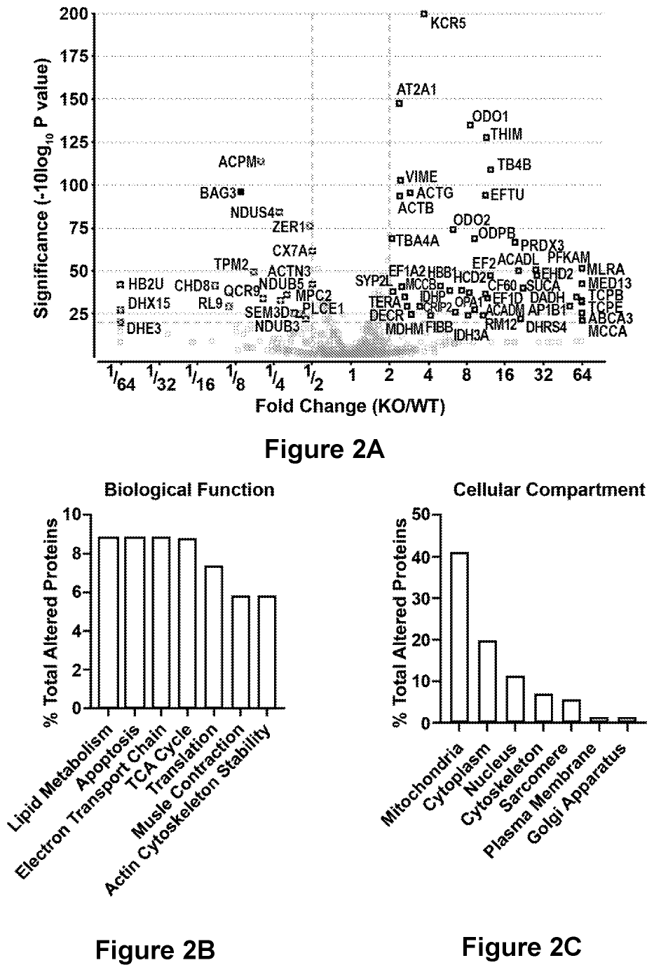

Protein from the mouse left ventricle was digested with trypsin protease and the resulting peptides were subjected to high pressure liquid chromatography coupled to tandem mass spectrometry (LC-MS/MS). The acquired spectra were then searched against the Mus musculus database and analyzed using label free quantification (LFQ) normalized by total ion current. LFQ analysis identified 86 proteins with significantly altered expression in the BAG3 KO mice (p<0.05 vs. WT, FIG. 2A and Table 1 below).

| | | Significance |
|---|---|---|
| Uniprot Accession | Gene Name | (−101og₁₀P value) |
| Q6P8J7\|KCRS_MOUSE | Ckmt2 | 200 |
| Q8R429\|AT2A1_MOUSE | Atp2a1 | 147.5 |
| Q60597\|ODO1_MOUSE | Ogdh | 135.11 |
| Q8BWT1\|THIM_MOUSE | Acaa2 | 127.61 |
| Q9CR21\|ACPM_MOUSE | Ndufab1 | 113.31 |
| P68372\|TBB4B_MOUSE | Tubb4b | 109.01 |
| P20152\|VIME_MOUSE | Vim | 102.81 |
| Q9JLV1\|BAG3_MOUSE | Bag3 | 96.01 |
| P63260\|ACTG_MOUSE | Actg1 | 95.67 |
| Q8BFR5\|EFTU_MOUSE | Eef1a1 | 93.95 |
| P60710\|ACTB_MOUSE | Actb | 93.24 |
| Q9CXZ1\|NDUS4_MOUSE | Ndufs4 | 84.27 |
| Q80ZJ6\|ZER1_MOUSE | Zer1 | 75.78 |
| Q9D2G2\|ODO2_MOUSE | Dlst | 74.08 |
| P68368\|TBA4A_MOUSE | Tuba4a | 69 |
| Q9D051\|ODPB_MOUSE | Pdhb | 68.49 |
| P20108\|PRDX3_MOUSE | Prdx3 | 66.6 |
| P56392\|CX7A1_MOUSE | Cox7a1 | 61.34 |
| Q9QVP4\|MLRA_MOUSE | Myl7 | 51.3 |
| P47857\|PFKAM_MOUSE | Pfkm | 50.86 |
| P51174\|ACADL_MOUSE | Acadl | 49.86 |
| P58774\|TPM2_MOUSE | Tpm2 | 49.64 |
| P58252\|EF2_MOUSE | Eef2 | 47.39 |
| Q8BH64\|EHD2_MOUSE | Ehd2 | 47.27 |
| Q5SWW4\|MED13_MOUSE | Med13 | 42.57 |
| O88990\|ACTN3_MOUSE | Actn3 | 42.14 |
| P06344\|HB2U_MOUSE | | 41.86 |
| P02088\|HBB1_MOUSE | Hbb-b1 | 41.34 |
| Q09XV5\|CHD8_MOUSE | Chd8 | 41.26 |
| P62631\|EF1A2_MOUSE | Eef1a2 | 40.71 |
| Q9WUM5\|SUCA_MOUSE | Suclg1 | 39.93 |
| O08756\|HCD2_MOUSE | Hsd17b10 | 38.94 |
| P54071\|IDHP_MOUSE | Idh2 | 38.74 |
| Q8BWB1\|SYP2L_MOUSE | Synpo2l | 38.06 |
| P58281\|OPA1_MOUSE | Opa1 | 37.3 |
| P63038\|CH60_MOUSE | Hspd1 | 36.61 |
| Q9CQH3\|NDUB5_MOUSE | Ndufb5 | 35.82 |
| Q3ULD5\|MCCB_MOUSE | Mccc2 | 34.62 |
| O08749\|DLDH_MOUSE | Dld | 34.37 |
| P57776\|EF1D_MOUSE | Eef1d | 33.75 |
| Q8R1I1\|QCR9_MOUSE | Uqcr10 | 33.65 |
| P80314\|TCPB_MOUSE | Cct2 | 32.85 |
| Q9D023\|MPC2_MOUSE | Mpc2 | 32.43 |
| P80316\|TCPE_MOUSE | Cct5 | 31.88 |
| Q9CQ62\|DECR_MOUSE | Decr1 | 29.53 |
| O35643\|AP1B1_MOUSE | Ap1b1 | 29.47 |
| P51410\|RL9_MOUSE | Rpl9 | 29.43 |

Notably, a pathway analysis of these proteins revealed that the largest proportion (36%) are primarily localized in mitochondria (FIG. 2B), suggesting disruption of mitochondrial function may contribute to the cardiac dysfunction associated with reduced Bag3 expression. When 86 proteins were analyzed by their biological function, among their primary functions were regulating mitochondrial metabolism and mitochondria-dependent apoptosis (FIG. 2C). Bag3 has been linked to mitochondrial function in neonatal rat ventricular cardiomyocytes, where Bag3 knockdown was associated with reduced mitophagy (39). However, this is the first indication that reducing Bag3 severely alters expression of mitochondrial proteins and further indicates a role for Bag3 in mitochondria-mediated cell survival pathways and non-mitochondrial inflammation in the heart.

### The Stress of Hypoxia-Reoxygenation: Apoptosis in BAG3+/−Mice

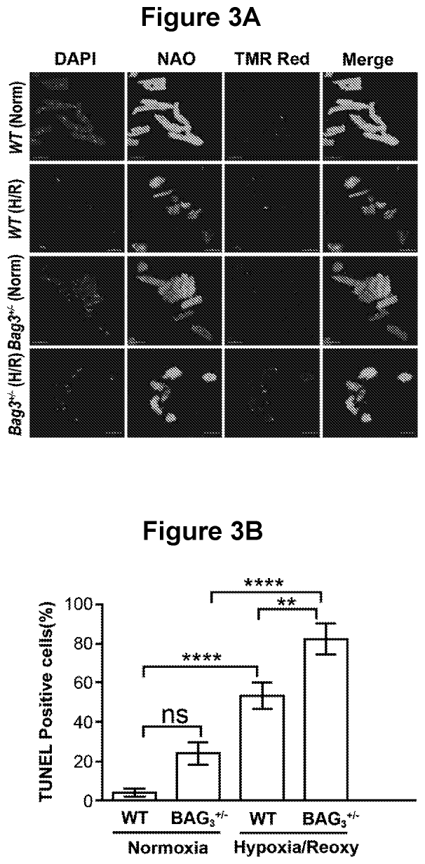

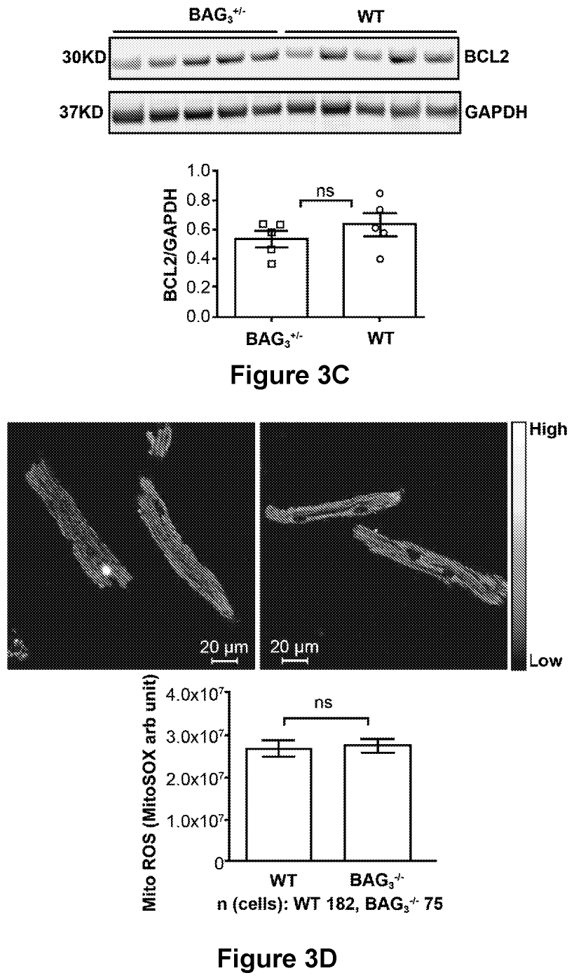

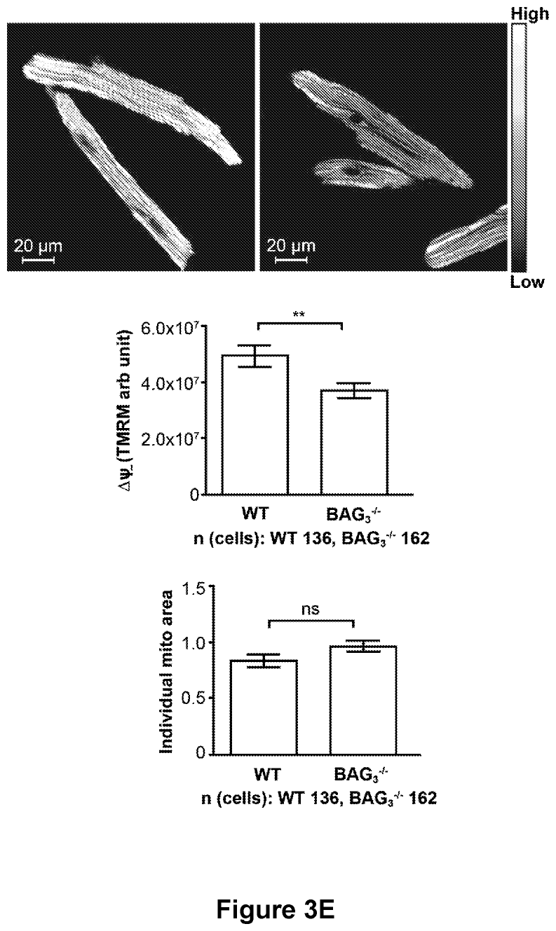

To determine how Bag3 haplo-insufficiency influences apoptosis in the mouse model of BAG3 deficiency, adult myocytes were harvested from 8 to 10 week old WT and BAG3^{+/-} mice and stained them for nuclear DNA (DAPI), viable mitochondria (NAO) and damaged DNA (TMR Red-TUNEL), and the resulting confocal images analyzed (FIG. 3A) with one-way analysis of variance, followed by sub-group analysis with Bonferroni correction (FIG. 3B; p<0.0125 is statistically significant). In the absence of stress, there was a very small and non-significant (p=0.1130) increase in apoptosis in the Bag3^{+/-} mice. When WT myocytes were stressed with one hour of hypoxia and subsequent re-oxygenation (H/R) for two hours, there were significantly (p<0.0001) more TUNEL positive cells when compared to WT-normoxic cells; as expected after H/R injury. Similarly, H/R resulted in significantly (p<0.0001) more TUNEL positive Bag3^{+/-} myocytes when compared to Bag3^{+/-}-normoxic cells. More interestingly, H/R resulted in significantly (p=0.0076) more TUNEL positive cells in Bag3^{+/-} myocytes when compared to WT-H/R myocytes; indicating that BAG3 haplo-insufficiency exacerbates H/R injury.

### Homozygous Loss of BAG3 Alters the Mitochondrial Membrane Potential (ΔΨₘ)

A separate group of studies were undertaken to use confocal microscopy to measure the effects of Bag3 deletion on mitochondrial area (size) and the level of reactive oxygen species (ROS). Because pilot experiments suggested that heterozygous BAG3 deletion had no effect on mitochondrial reactive oxygen species, cells isolated from Bag3^{-/-} mice were used for these experiments but planned to follow-up positive studies. As seen in FIG. 3D, homozygous deletion had no effect on mitochondrial reactive oxygen species, nor did it have an effect on mitochondrial content. By contrast, when these same cells were stained with a marker of membrane potential, there was a significant (p<0.01) decrease in the mitochondrial membrane potential (ΔΨₘ) in Bag3^{-/-} mice when compared with WT mice (FIG. 3E).

Comparative Analysis of Relevant Protein Levels in Bag3^{+/-} and Bag3^{WT} Mice

To confirm the results of the proteomic studies, levels of proteins were measured that are critical for myocyte homeostasis and proteostasis in left ventricular myocardium of the Bag3^{WT} and Bag3^{+/-} mice with a focus on apoptosis-related proteins. The intrinsic pathway of apoptosis is activated by signals derived from mitochondria.

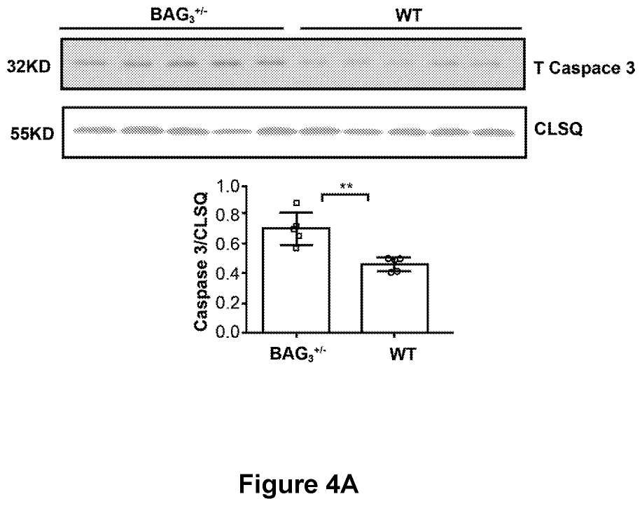

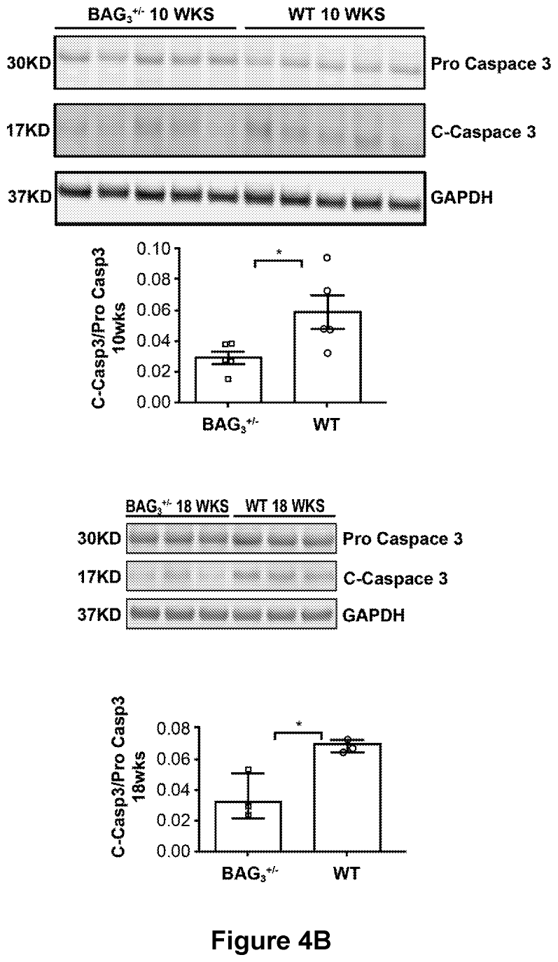

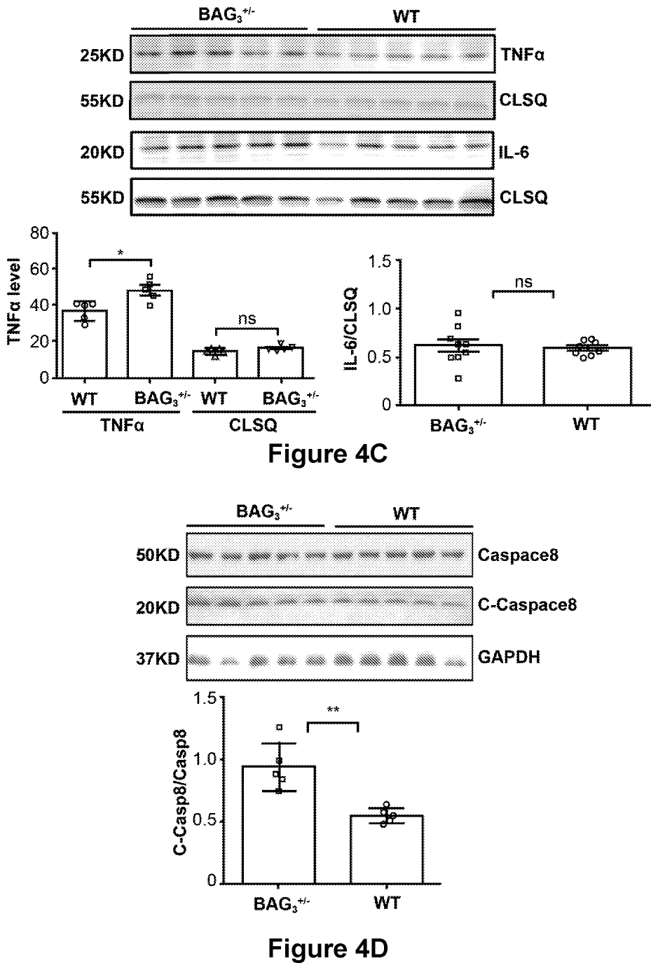

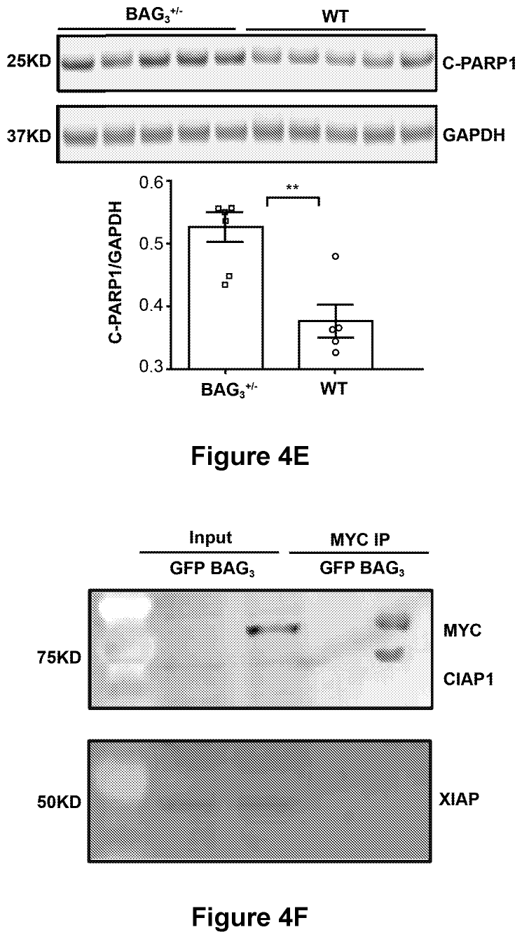

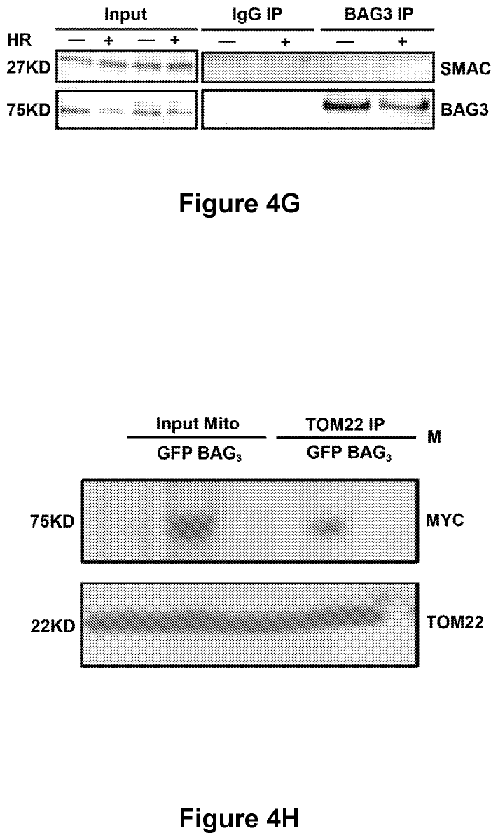

As seen in FIG. 4A, there was a significant (p<0.01) increase in the levels of caspase 3, a primary executioner (effector) protein of apoptosis. However, the ratio of cleaved caspase 3/total caspase 3 protein was not elevated in the ventricular myocardium from Bag3^{+/-} mice when compared with protein isolated from the hearts of WT mice suggesting that Bag3 haplo-insufficiency causes only modest to minimal apoptosis at the early stage of the disease. Although not wishing to be bound by any theory or hypothesis, while activation of caspase-3 was observed with early disease when cardiac remodeling was absent, there may be activation of caspase 3 in older mice with cardiac remodeling and diminished function. However, in older mice at either 10-12 weeks of age or at 18-22 weeks of age there was a significant increase in the levels of activated caspase-3, but the ratio of cleaved caspase 3/total caspase 3 protein still was not increased (compared to WT) suggesting that only profound stress coupled with old age would activate caspase-3-mediated apoptosis in the murine heart (FIG. 4B).

### BAG3 Deficiency and the Extrinsic Pathway of Apoptosis

The extrinsic/mitochondrial independent pathway that is activated by tumor necrosis factor-alpha (TNFa) through binding to the TNFR1 receptor with the subsequent cleavage and activation of caspase 8 (40) was then evaluated. Cleaved caspase 8 can then activate caspase 3 which leads directly to apoptosis or alternatively it can bind to bid, a member of the Bcl-2 family, which then interacts with tBid which after translocating into the mitochondria initiates the release of cytochrome c. Cytochrome c then binds to the apoptosome with subsequent activation of pro-caspase 9.

The extrinsic pathway was clearly activated in the Bag3^{+/-} mice, as levels of TNFa were significantly higher in Bag3^{+/-} mice (58.0±2.7, n=5; p=0.014) when compared with WT littermate control mice (36.7 4 2.7; n=5) (FIG. 4C). These effects were TNF-restricted as no change in levels of pro-inflammatory cytokine IL-6 was detected (FIG. 4C). The role of extrinsic signaling in apoptosis in Bag3^{+/-} mice was further supported by the finding that the ratio of cleaved caspase 8/total caspase 8 (0.94±0.09, n=5) was significantly (p<0.003) increased in Bag3^{+/-} ventricular myocardium when compared with tissue obtained from WT mice. (0.54±0.03, n=5, FIG. 4D).

That the decrease in the levels of Bag3 were associated with an increase in cellular inflammation was supported by the finding that Bag3^{+/-} hearts had a significant increase of poly(ADP-ribose) polymerase-1 (PARP-1), (FIG. 4E) a protein that transfers ADP-ribose to apoptosis-inducing factor (AIF), resulting in its translocation from mitochondria to the nucleus where it initiates cell death by signaling DNA fragmentation (41,42). Thus, in aggregate, these studies of protein levels in myocardial cells from mice in which one allele of BAG3 was deleted indicate that canonical TNF-receptor signaling is a characteristic feature of Bag3 haploinsufficiency and that it leads to the activation of PARP1 and AIF which in turn results in sterile inflammation and DNA fragmentation (41,42).

Caspases, SMAC and cIAP—Control of Apoptotic Signaling

To better understand the mechanisms of Bag3's regulation of apoptosis and mitochondrial homeostasis, proteins that were part of the Bag3 proteome were examined. As already noted, caspase 3 is one of the executioner caspases that sits at the terminal end of the apoptotic signaling cascade and its activity is dependent on signals that involve the mitochondria, including the release of cytochrome c and endonuclease G from the mitochondrial matrix. It is therefore considered to be a “mitochondrial dependent” caspase. In the absence of stress, the cell inhibitor of apoptosis (cIAP) binds to a receptor on caspase 3 (as well as all other caspases) and inhibits its ability to be cleaved into the active moiety. However, in the presence of stress, e.g., ischemia or toxin, the second mitochondrial derived activator of caspase (SMAC) is released from the outer mitochondrial membrane (OMM), (and other caspases). Caspase 3 is then cleaved and can begin to effect apoptosis of cardiac cells.

The data in the young Bag3^{+/-} mice presented a conundrum: how could apoptosis be clearly increased in adult myocytes from Bag3^{+/-} mice, while the ratios of cleaved/pro caspase 3 were unchanged. A possible explanation for this apparent discrepancy is potential changes in the function of SMAC. SMAC, a protein transcribed by the DIABLO gene is located in the inter-mitochondrial space. As seen in FIG. 4F, for the first time cIAP co-immuno-precipitates (co-IP's) with Bag3 and with caspase-3 but not with SMAC, and Bag3 co-IP's with cIAP but not with SMAC or with caspase 3. The relationship between Bag3 and cIAP was selective, as Bag3 does not co-immuno-precipitate with the highly homologous XIAP (x-linked IAP) (FIG. 4F).

A second important element in modulating the homeostasis of the mitochondria is the protein import system called the translocase of the outer membrane (TOM) and the translocase of the inner membrane system called TIM. TOM and TIM proteins are required for the translocation of key elements into and out of mitochondria. TOM22 (along with TOM20) is an accessory unit of the Translocator of Outer Membrane (TOM), a pore in the OMM that transports short chains of proteins into the mitochondrial matrix. Interestingly, Bag3 also co-immuno-precipitates with the TOM22 protein (FIG. 4F).

### SMAC is Stuck in the OMM in the Absence of Bag3

Although not wishing to be bound by any theory or mechanism of action, one hypothesis was that caspase-3 was not activated with Bag3 haplo-insufficiency due to SMAC being stalled in the mitochondrial membrane and not re-localizing to the cytoplasm. To address this hypothesis, neonatal myocytes from wild type mice were isolated and cultured. The cells were then exposed to: 1) normal control conditions 2) hypoxia/re-oxygenation; 3) siRNA for Bag3; or 4) hypoxia/reoxygenation plus Bag3 siRNA.

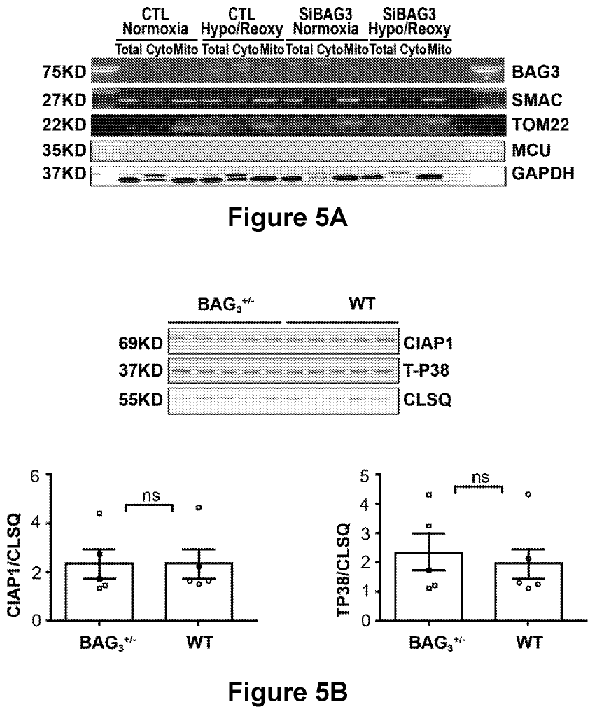

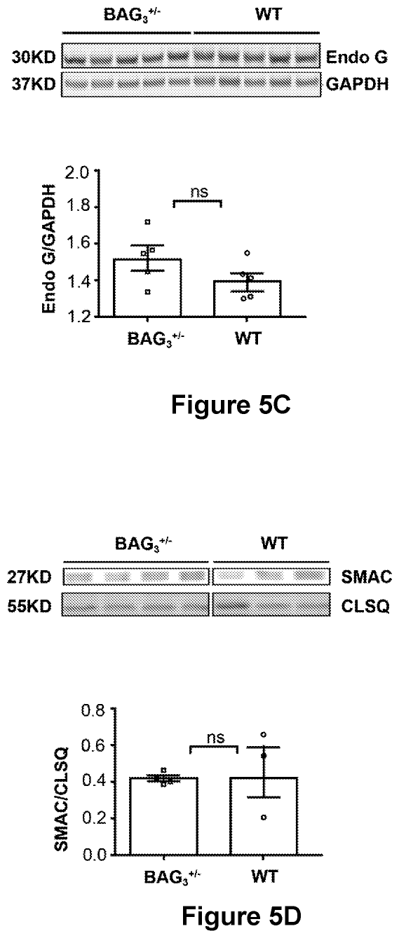

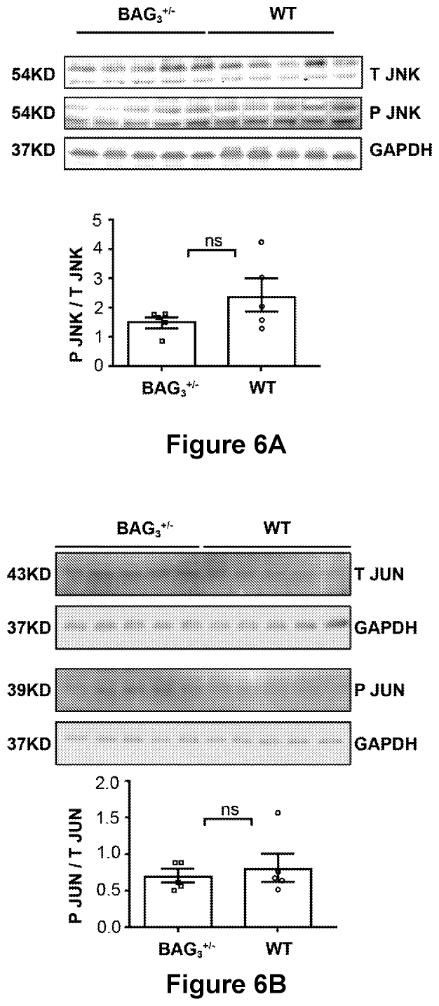

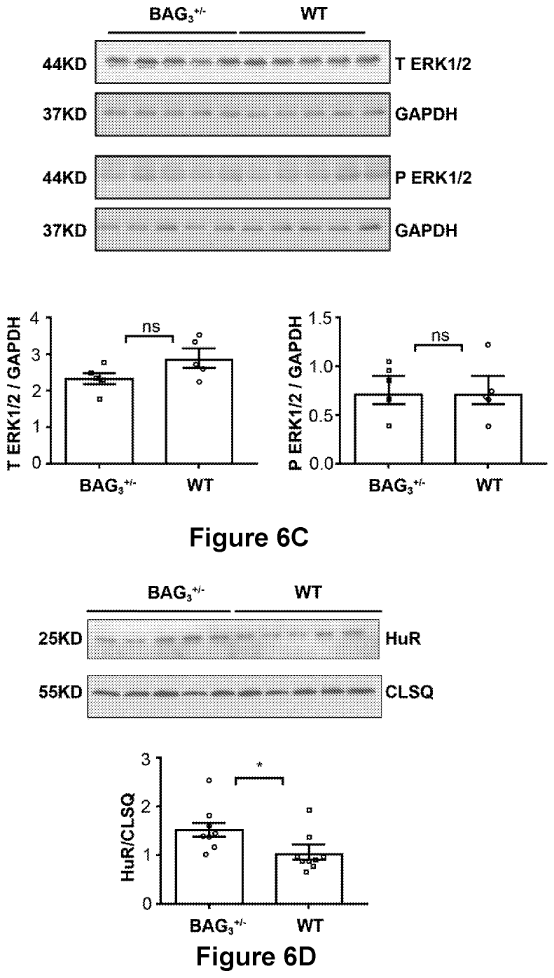

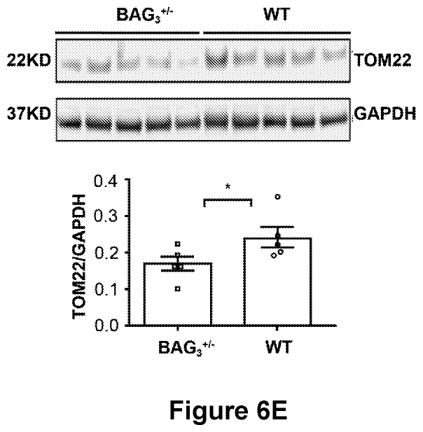

As seen in FIG. 5A, under control culture conditions, SMAC was prominent in the mitochondria with a small amount of protein in the cytoplasm. The addition of H/R stress on its own did not change this localization of SMAC. However, in the absence of Bag3 (siRNA Bag3) there was no appreciable SMAC in the cytoplasm. Similarly, no SMAC was seen in the cytoplasm when cells lacking Bag3 (siBag3) were exposed to the stress of H/R. TOM22 served as a control because it is exclusively mitochondrial. It remained in the mitochondria throughout the four study conditions indicating that the translocation of SMAC requires Bag3.

### The BAG3^{+/-} Pre-Heart Failure Proteome

To gain a broader understanding of how Bag3 depletion impacts apoptosis, other apoptotic signaling proteins that are associated with myocardial failure were assessed. FIG. 5 shows that the effects of Bag3 deficiency in the 8-week-old Bag3^{+/-} mice differ substantially from the changes in protein levels that are the hallmarks of LV dysfunction seen in later stages of disease. For example, no change in either cIAP or Total-P39, a MAP kinase that is implicated in mediating the pathologic changes accompanying inflammatory and apoptotic processes (43) was observed (FIG. 5B). As seen in FIGS. 5A thru 5C, no change was observed in JNK which is upregulated in the failing heart and activates multiple pro-apoptotic signaling pathways or in Jun which is differentially regulated in HFrEF and has a very similar effect on the cell in part by releasing SMAC from the mitochondria. Finally, expression of phosphor-ERK1/2 and Total-ERK was assessed, but there were no changes attributable to BAG3.

One observation that derived from the proteomic screens was unexpected but consistent with the over-arching hypothesis that abnormal levels of Bag3 led to an increase in the cardiac inflammasome. The RNA-binding protein Human Antigen R (HuR: ELAV-1) was over-expressed in the Bag3^{+/-} hearts (1.51±0.16, n=8) when compared with levels in WT control mice (1.07±0.11, n=9, p=0.03) (FIG. 5D). This might not be surprising in light of the observation that Bag3 haplo-insufficiency is associated with an increase in the activity of the inflammasome as HuR knockdown attenuates the inflammatory response and might be a therapeutic target in pathological cardiac hypertrophy (44,45). Thus, in aggregate, these studies of protein levels in myocardial cells from mice in which one allele of Bag3 was deleted strongly suggest that canonical TNF-receptor signaling is a characteristic feature of Bag3 haploinsufficiency and that it leads to inflammation and DNA fragmentation. There was also a significant decrease in the levels of TOM 22, a protein that regulates the uptake of proteins and other nutrients into the mitochondrial matrix (FIG. 4E).

### Bag3 Haplo-Insufficiency Causes Abnormal Mitochondrial Ca²⁺ Homeostasis

As seen in FIGS. 2A thru 2C and in Table 1, the proteomic studies disclosed herein demonstrated that a primary effect of Bag3 haplo-insufficiency was a change in the amount of specific mitochondrial proteins that functioned in cellular metabolism and energy production. Specifically, the knock-out of one BAG3 allele resulted in decreased expression of enzymes associated with mitochondrial function including isocitrate dehydrogenase, pyruvate dehydrogenase and alpha ketoglutarate dehydrogenase. Three investigational approaches were therefore undertaken to evaluate the hypothesis that haplo-insufficiency of Bag3 could be a causative factor in the diminished function of Bag3^{+/-} in hearts because it led to abnormal mitochondrial Ca²⁺ homeostasis.

### 1) Haplo-Insufficiency of Bag3 Impedes the Ability of the Heart to Maintain the Mitochondrial Membrane Potential (MMP)

In the first set of studies, adult myocytes were extracted from WT and Bag3^{+/-} mice. The myocytes were then exposed to H/R and mitochondrial membrane potential (ΔΨₘ) was evaluated using the ratiometric indicator JC-1 and mitochondrial Ca²⁺ uptake using ratiometric dye Fura-FF. Fluorescence was measured using a dual wavelength-emission spectrofluorometer as described previously (35).

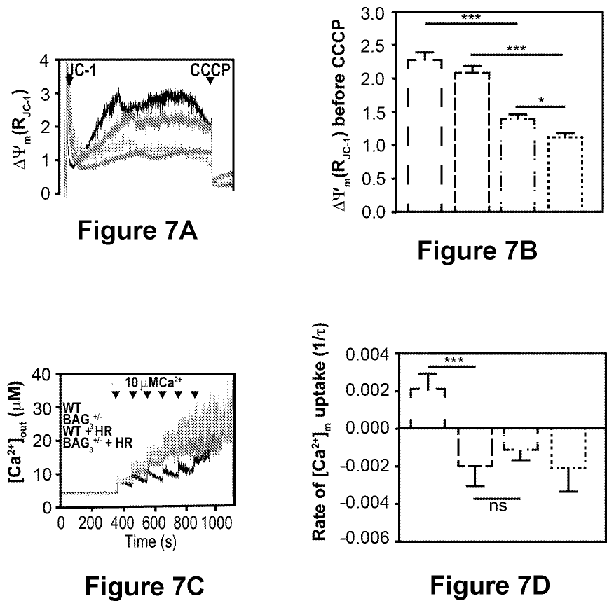

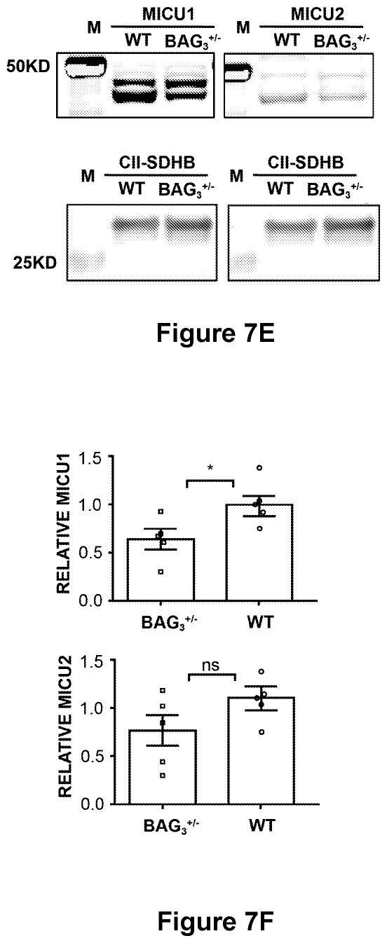

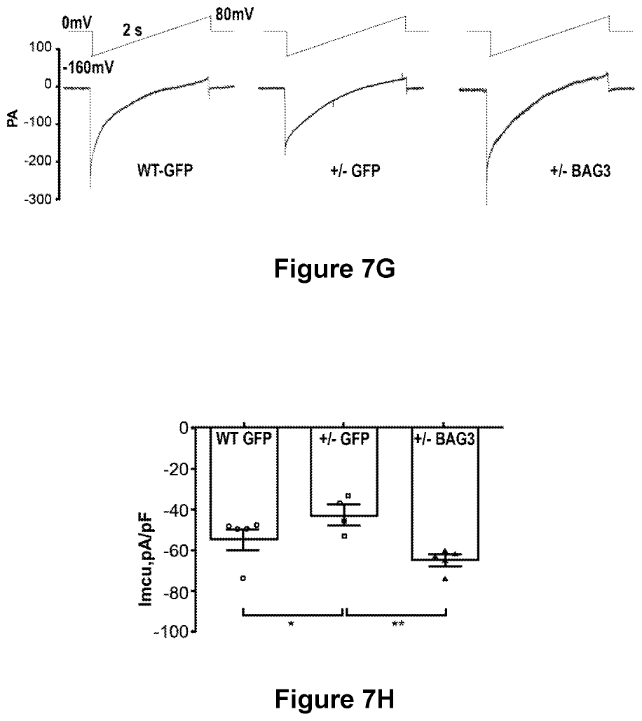

As seen in FIGS. 7A and 7B, there was a significant (p<0.001) decrease in the ΔΨₘ in myocytes from normoxic WT hearts when compared with myocytes from WT hearts that were exposed to the stress of H/R as expected. There was also a significant reduction (p<0.001) in ΔΨₘ in myocytes from Bag3^{+/-} mice when compared with myocytes from Bag3^{+/-} mice after H/R However, there was an additional significant (p<0.05) reduction in ΔΨₘ in BAG3^{+/-} myocytes when compared with WT myocytes exposed to H/R suggesting that the stress of H/R aggravated the underlying effects Bag3 haplo-insufficiency on mitochondrial function. A similar phenomenon was observed when the effect of the deletion of one allele of Bag3 on calcium homeostasis was looked at (FIGS. 7C and 7D). When adult myocytes from WT mice were compared with cells isolated from Bag3^{+/-} mice there was a significant (p<0.001) decrease in the rate of [Ca²⁺]ₘ uptake (lit) by Bag3^{+/-} mitochondria when compared with mitochondria from WT mice. In aggregate, these results support the hypothesis that Bag3 depletion results in substantial changes in Ca²⁺ homeostasis, especially in cells that are stressed.

### 2) Heterozygous Deletion of Bag3 Alters the Function of the Ca²⁺ Uniporter.

The uniporter is composed of two pore-forming subunits (MCU and MCUb) and three regulatory subunits (MICU1, MICU2, and EMRE) which together maintain the negative potential of the outer mitochondrial membrane (OMM) (46). This negative potential across the OMM is responsible for the movement of valuable resources into the mitochondria. Under resting conditions, MICU1 and MICU2 dimerize and serve as gatekeepers for the MCU. The initiation of cytosolic [Ca²⁺] release into the mitochondria causes a conformational change in the protein complex by blocking MICU-2 dependent inhibition of Ca²⁺ movement. MICU1 activates the channel and stimulate Ca²⁺ transport into the mitochondria. EMRE stabilizes the MCU-MICU1 complex which in turn fine tunes the level of Ca²⁺ that can enter the mitochondria.

As seen in FIGS. 7E and 7F, there was a trend towards a decrease in the level of MICU2 in Bag3^{+/-} mice when compared with Bag3-WT mice, but this trend did not reach statistical significance. There was, however, a significant (p<0.05) decrease in the levels of MICU1 in Bag3^{+/-} mice when compared with the WT controls providing further support that BAG3 haplo-insufficiency is directly associated with and causative of the development of abnormalities in mitochondrial Ca²⁺ homeostasis.

### 3) MCU Function is Normalized by Adenovirus Over-Expression of Bag3

To confirm that decreased mitochondrial calcium uptake was due to reduced MCU activity and was directly related to the haplo-insufficiency in Bag3, cardiac mitoplasts were isolated from WT myocytes and from Bag3^{+/-} myocytes (both overexpressing GFP as a control). Currents (I_{MCU}) were measured from voltage-clamped mitoplasts before and after addition of 5 mM Ca²⁺. These measurements, though technically challenging, allow comparisons of MCU activity between different groups of mitochondria by tightly controlling conditions of membrane potential, Ca²⁺ and H⁺ gradients. I_{MCU} were recorded during a voltage ramp as indicated.

As shown in FIGS. 7G and 7H, peak I_{MCU} in WT-GFP mitoplasts was significantly (p=0.018) higher than Bag3^{+/-} GFP mitoplasts (n=5 each). Importantly, adenovirus-mediated overexpression of WT Bag3 in Bag3^{+/-} myocytes restored peak I_{MCU} to normal (p=0.028 when compared to Bag3^{+/-}). These data indicate that adequate levels of BAG3 are necessary to maintain MCU expression and/or activity.

The Human Proteome and Inflammasome Reflects that Seen in Bag3^{+/-} Mice

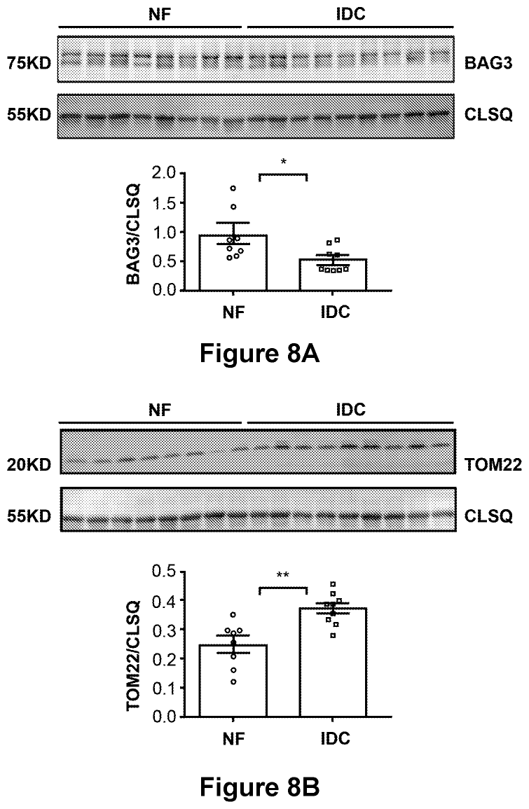

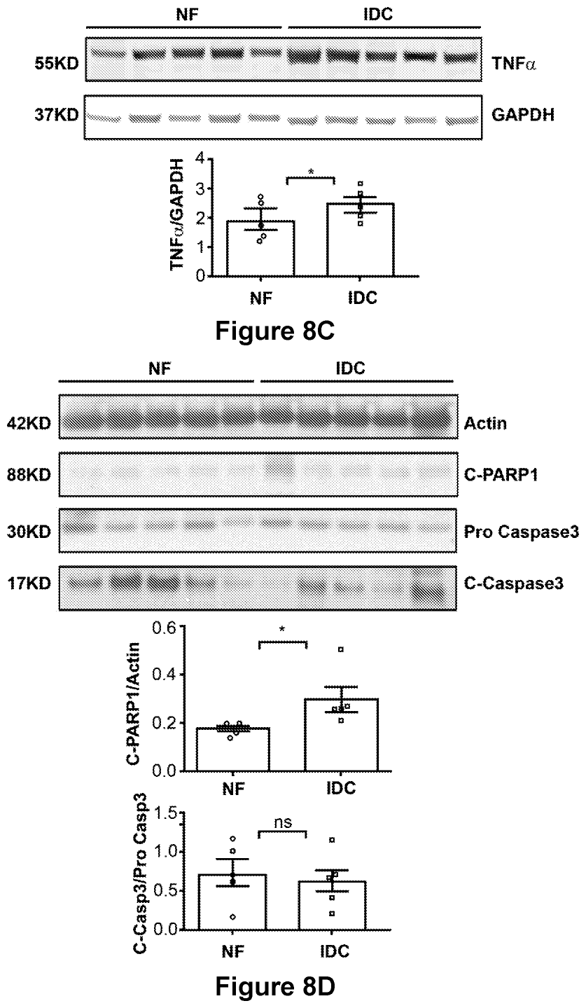

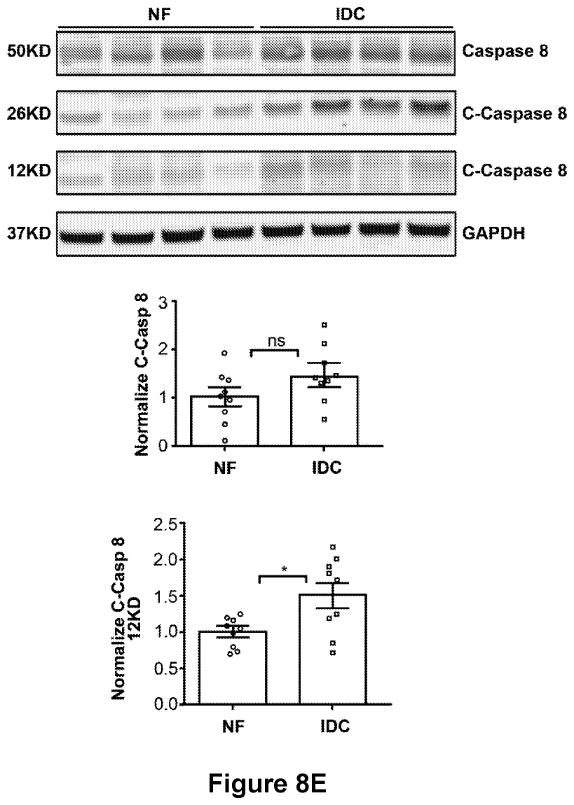

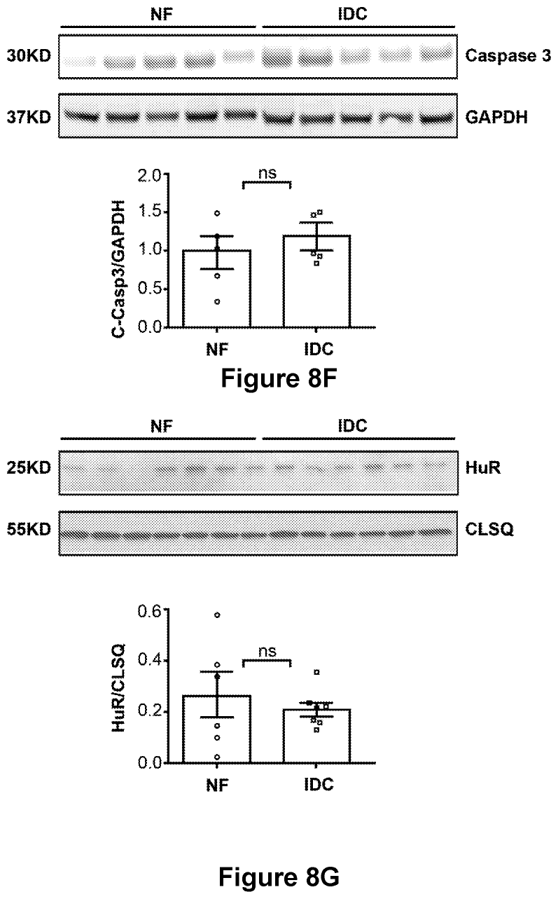

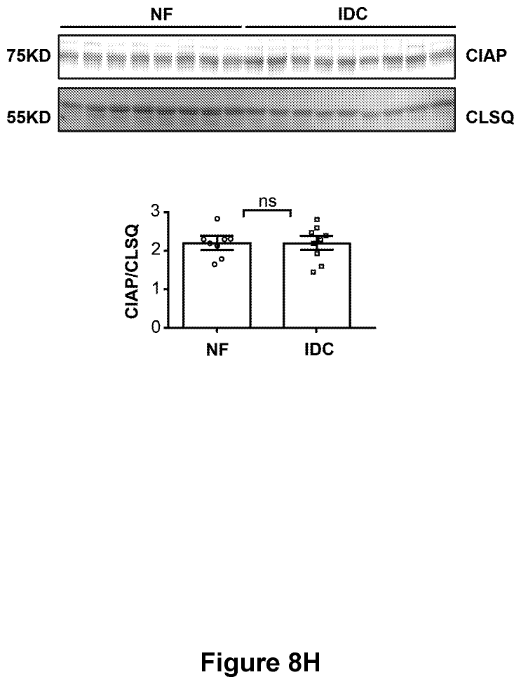

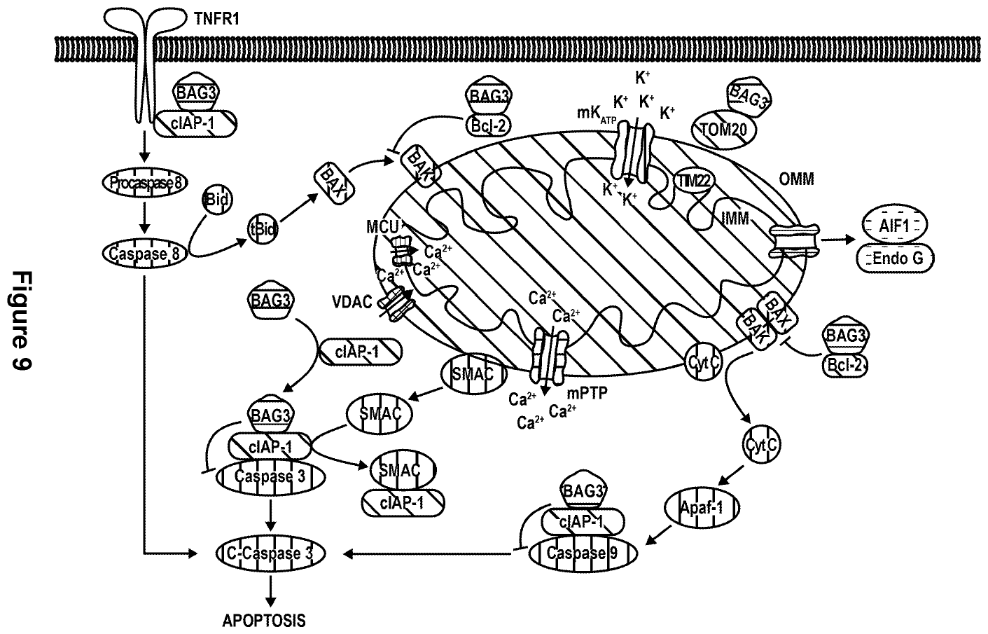

As seen in FIG. 8A, levels of Bag3 are reduced by approximately 50% in LV myocardium of failing human heart tissue when compared with non-failing controls. Furthermore, the inflammasome of the human heart was found to be similar but not identical to that in mice with haplo-insufficiency of Bag3: levels of cPARP and cleaved caspase 8 are elevated whereas levels of cleaved caspase 3 are not increased (FIGS. 8D and 8E). An increase in cPARP was also observed (FIG. 8D). By contrast with the mice, no significant change in HuR was observed (FIG. 8G).

### Example 3: Discussion

Just a little over a decade ago, Selcen and colleagues first reported that a sporadic single nucleotide polymorphism (SNP) in BAG3 that resulted in the substitution of a leucine for a proline at amino acid position 209 led to a phenotype that included giant axons, severe skeletal muscle dis-array and mild cardiac hypertrophy in children. Subsequent genome-wide association studies (GWAS) and whole exome (WES) and whole genome (WGS) sequencing have identified truncations and SNPs in BAG3 as being causative of a variety of cardiac phenotypes—most notably DCM (3-6). While young in age from a scientific knowledge perspective, BAG3 is quite old from a biologic standpoint as evidenced by the fact that Bag3 homologues have been found in plants (47). To date, genomic abnormalities in Bag3 have been associated at the protein and molecular levels with diminished autophagy, increased apoptosis, abnormal excitation-contraction coupling and abnormal sarcomere function. However, it was unclear whether the full extent of Bag3's functional capabilities are known.

Young mice in which a single allele of BAG3 was ablated but had not yet developed a decrease in ejection fraction and proteomics with high pressure liquid chromatography coupled to tandem mass spectrometry (LC-MS/MS) were used to identify previously unrecognized consequences of Bag3 haploinsufficiency. As shown in the proteomics study and subsequent investigation disclosed herein, BAG3 also plays a role in modifying the extrinsic pathways of apoptosis while also serving as a regulator of inflammation thru activation of the cardiac inflammasome and in particular the TNFR1 signaling cascade. As disclosed herein for the first time, Bag3 has been shown to play an equally important role in supporting transport of Ca²⁺ into and out of the mitochondria. This transport of Ca²⁺ maintains the mitochondrial membrane potential that is required for Ca²⁺ flux—the biological event that provides the energy required by the enzymes of the tri-carboxylic acid (TCA) cycle.

The cellular machinery that enables Bag3 to remove inextricably damaged or diseased organelles and cells from tissues without the collateral damage that occurs when cells die suddenly is complex and involves multiple regulatory pathways. When cell death is programmed as in apoptosis, the cell membrane remains intact throughout the process such that internal enzymes and toxic materials are degraded prior to the ultimate end of the cell as a functioning organelle. As reported over a decade ago, two canonical pathways regulate apoptosis in the heart: the type I or the type II pathway (48). The Type 1 (extrinsic or mitochondrial independent) pathway, consists of a cascade of events that begins with activation of a death domain receptor such as the tumor necrosis factor receptor-1 (TNFR-1) and ends with activation of the executioner caspases-7 and -3. By contrast, in the Type II (intrinsic or mitochondrial-dependent) pathway, apoptosis is activated by the release of pro-apoptotic signals from the mitochondria including cytochrome c and endonuclease g and the subsequent activation of caspase-9 and -3.

Apoptotic cell death was proposed to not necessarily occur as a direct result of the activation of the cell death pathways but is due instead to sustained TNF signaling that leads to cell death after one or more anti-apoptotic proteins become depleted whereas over-expression of Bcl-2 was sufficient to partially attenuate this pathway (42). However, when this model was proposed over a decade ago, BAG3 had only recently been discovered and its importance to the cell and the whole organism were completely unknown. Indeed, it was thought to regulate apoptosis by binding to Bcl2 and link the actin filaments with the Z disc. In fact, an early paper erroneously suggested that haplo-insufficiency of BAG3 would not result in a HF phenotype. It is now known, however, that the absence of a full complement of the strongly anti-apoptotic Bag3 protein resulted in a very similar phenotype to the Bag3 haplo-insufficiency model with an increase in total caspase 3 activity and caspase 8 activity. What is unique to the haplo-insufficiency of Bag3 model is an increase in the levels of TNF as well as an adverse change (decrease) in the mitochondrial membrane potential independent of exogenous TNF. Furthermore, the diminished role of cIAP due to its inability to partner with a full complement of Bag3 is likely a contributing factor in the pathobiology of BAG3 deficiency. Thus, an imbalance between apoptosis and survival has severe consequences in the myocardium.

In the canonical pathways of apoptosis, the Type 1 pathway is down-regulated by inhibitors of apoptosis (IAPs) (49). By contrast, the Type 2 pathway is inhibited by the binding of Bag3 to Bcl-2 (50), the founding member of the very large Bcl-2 family of proteins that includes both pro-apoptotic (BIM, BID, BAD, BAX/BAK) and anti-apoptotic (Bcl-2, Bcl-X_{L}, MCL-1) members (50). As disclosed herein, the loss of one Bag3 allele and the resulting decrease in Bag3 levels by 50% was associated with an increase in caspase 3 but not a change in the ratio of cleaved caspase 3/total caspase-3. Rather, there is a significant increase in caspase-8 and cleaved caspase 8 and an increase in the ratio of cleaved caspase 8 to total caspase 8 which is associated with an increase in apoptosis. Once activated, caspase 8 is responsible for apoptosis induced by the death receptors Fas, TNFR1 and DR3. In fact, in previous studies, deletion of caspase-8 and RIPK3 prevent aberrant cell death, reduce the inflammation and prolong mouse survival (49). Caspase-8 has been studied extensively in cancer but less so in the heart (49,51,52). However, the results of these studies suggests that caspase-8 plays a greater role in the heart than has been appreciated.

One of the interesting and novel observations from the studies herein is that cIAP co-immuno-precipitates with Bag3. This association between Bag3 and cIAP is selective as it was found that Bag3 does not precipitate with the x-linked homolog XIAP, an inhibitor that directly neutralizes caspase-9 and the effector caspases-3 and -7 and is found in abundance in many different types of advanced cancers (53). Previous studies in inflammatory bowel disease and in cancer have shown that cIAPs and their antagonists regulate spontaneous and TNF-induced proinflammatory cytokine and chemokine production (54). Since BAG3 immuno-precipitates with cIAP, and cIAP has previously been shown to couple with the TNFR1 at the critical junction of TRAF2/TRAF5, LUBAC and RIPK1, the presence or absence of Bag3 is likely to have an important role in TNF signaling and thus in the inflammasome of myocardial cells (55).

Surprisingly, Bag3 did not co-precipitate with either caspase-3 or SMAC. When SMAC translocates to the cytoplasm it competes with cIAP, dislodging it and allowing caspase-3 to be activated. Although the studies herein suggest that this translocation does not occur in the absence of Bag3, further studies can elucidate the mechanism by which BAG3 regulates SMAC translocation especially in view of the observation that Bag3 and SMAC did not physically associate with each other.

The mitochondrial Ca²⁺ (mCa²⁺) uniporter, the major pathway for Ca²⁺ uptake by the mitochondria, plays an important role in the heart as it is responsible for ATP production by the tri-carboxylic acid cycle and in particular the activity of the Ca²⁺ regulated enzymes of the tri-carboxylic acid cycle (56). In fact, the proteomic studies of Bag3^{+/-} mice showed that Bag3 is associated with an increase in the Ca²⁺-dependent tri-carboxylic enzymes including pyruvate dehydrogenase, alpha ketoglutarate dehydrogenase and isocitrate dehydrogenase (57). Supra-normal levels of mCa²⁺ result in an increased workload thereby increasing cell stress whereas the opposite occurs when mCa²⁺ levels are low and stress is minimized (58). The MCU is the major pathway for Ca²⁺ uptake by the mitochondria whereas the mitochondrial permeability transition pore is the site through which excess Ca²⁺ is lost.

While it would be attractive to suggest that the decrease in MCU level and activity in Bag3^{+/-} hearts contributes to the HFrEF phenotype, the biology of mCa²⁺ is complex and not without controversy. For example, mitochondrial calcium uniporter inhibition was purported to be beneficial in both cardiac hypertrophy and in heart failure with reduced function.(59,60) By contrast, studies in zebra fish(61), diabetic mouse heart(62) and in a guinea pig model of heart failure(58) showed that restitution of normal or supra-normal levels of MCU would have salutary effects on heart function. The mechanisms responsible for the molecular and cellular biology of the mitochondria, particularly how it relates Ca²⁺ homeostasis, has also been debated (58,63)-(64). In view of the controversy, the observation that restoration of normal levels of Bag3 improves mitochondrial Ca²⁺ uptake and by extension enhances cellular bioenergetics, thereby providing benefit to the cell should be interpreted carefully.

Bag3 is ideally suited to the role of multi-tasking in view of its many protein-protein binding domains. However, unlike intracellular proteins that reside in specific domains of a cell such as receptors in the sarcolemma or contractile elements in the sarcomere, BAG3 is ubiquitous (FIG. 9). However, it appears to establish discrete intracellular micro-environments based on its specific responsibilities and locations. For example, it couples the contractile elements to the Z disc in the cardiac sarcomere, couples the b-adrenergic receptor to the L-type Ca²⁺ channel in the sarcolemma, connects the dynein motor protein to the peri-nuclear aggresomes and co-chaperones the protein constituents of autophagy within the domain of the proteasome.

The results disclosed herein demonstrate that BAG3 also co-locates with TOM22 in the mitochondria. Thus, rather than simply being a regulatory or even a structural protein, Bag3 appears to serve as a universal glue that selectively localizes proteins to specific cellular domains where it can interact with and perhaps co-localize partner proteins. While not common, there are examples of this type of protein multi-tasking: the multi-functional protein 4.1R being a good example (65).

### REFERENCES

1. Behl C. Breaking BAG: The Co-Chaperone BAG3 in Health and Disease. Trends Pharmacol Sci 2016; 37:672-688.
2. De Marco M, Turco M C, Marzullo L. BAG3 in Tumor Resistance to Therapy. Trends Cancer 2020; 6:985-988.
3. Norton N, Li D, Rieder M J et al. Genome-wide studies of copy number variation and exome sequencing identify rare variants in BAG3 as a cause of dilated cardiomyopathy. Am J Hum Genet 2011; 88:273-82.
4. Shah S, Henry A, Roselli C et al. Genome-wide association and Mendelian randomisation analysis provide insights into the pathogenesis of heart failure. Nat Commun 2020; 11:163.
5. Choquet H, Thai K K, Jiang C et al. Meta-Analysis of 26 638 Individuals Identifies Two Genetic Loci Associated With Left Ventricular Ejection Fraction. Circ Genom Precis Med 2020; 13:e002804.
6. Mazzarotto F, Tayal U, Buchan R J et al. Reevaluating the Genetic Contribution of Monogenic Dilated Cardiomyopathy. Circulation 2020; 141:387-398.
7. Homma S, Iwasaki M, Shelton G D, Engvall E, Reed J C, Takayama S. BAG3 deficiency results in fulminant myopathy and early lethality. Am J Pathol 2006; 169:761-73.
8. McDermott-Roe C, Lv W, Maximova T et al. Investigation of a dilated cardiomyopathy-associated variant in BAG3 using genome-edited iPSC-derived cardiomyocytes. JCI Insight 2019; 4.
9. Fang X, Bogomolovas J, Wu T et al. Loss-of-function mutations in co-chaperone BAG3 destabilize small HSPs and cause cardiomyopathy. J Clin Invest 2017; 127:3189-3200.
10. Myers V D, Tomar D, Madesh M et al. Haplo-insufficiency of Bcl2-associated athanogene 3 in mice results in progressive left ventricular dysfunction, beta-adrenergic insensitivity, and increased apoptosis. J Cell Physiol 2018; 233:6319-6326.
11. Feldman A M, Begay R L, Knezevic T et al. Decreased levels of BAG3 in a family with a rare variant and in idiopathic dilated cardiomyopathy. J Cell Physiol 2014; 229:1697-702.
12. Toro R, Perez-Serra A, Campuzano O et al. Familial Dilated Cardiomyopathy Caused by a Novel Frameshift in the BAG3 Gene. PLoS One 2016; 11:e0158730.
13. Rosati A, Graziano V, De Laurenzi V, Pascale M, Turco M C. BAG3: a multifaceted protein that regulates major cell pathways. Cell Death Dis 2011; 2:e141.
14. De Marco M, Basile A, Iorio V et al. Role of BAG3 in cancer progression: A therapeutic opportunity. Semin Cell Dev Biol 2018; 78:85-92.
15. Sherman M Y, Gabai V. The role of Bag3 in cell signaling. J Cell Biochem 2021.
16. Hockenbery D, Nunez G, Milliman C, Schreiber R D, Korsmeyer S J. Bcl-2 is an inner mitochondrial membrane protein that blocks programmed cell death. Nature 1990; 348:334-6.
17. Feldman A M, Gordon J, Wang J et al. BAG3 regulates contractility and Ca(2+) homeostasis in adult mouse ventricular myocytes. J Mol Cell Cardiol 2016; 92:10-20.
18. Martin T G, Tawfik S, Moravec C S, Pak T R, Kirk J A. BAG3 Expression and Sarcomere Localization in the Human Heart are Linked to HSF-1 and are Differentially Affected by Sex and Disease. Am J Physiol Heart Circ Physiol 2021.
19. Kimura K, Ooms A, Graf-Riesen K et al. Overexpression of human BAG3(P209L) in mice causes restrictive cardiomyopathy. Nat Commun 2021; 12:3575.
20. Shy M, Rebelo A P, Feely S M et al. Mutations in BAG3 cause adult-onset Charcot-Marie-Tooth disease. J Neurol Neurosurg Psychiatry 2018; 89:313-315.
21. Quintana M T, Parry T L, He J et al. Cardiomyocyte-Specific Human Bcl2-Associated Anthanogene 3 P209L Expression Induces Mitochondrial Fragmentation, Bcl2-Associated Anthanogene 3 Haploinsufficiency, and Activates p38 Signaling. Am J Pathol 2016; 186:1989-2007.
22. Fang X, Bogomolovas J, Zhou P S et al. P209L mutation in Bag3 does not cause cardiomyopathy in mice. Am J Physiol Heart Circ Physiol 2019; 316:H392-H399.
23. Bristow M R, Minobe W A, Raynolds M V et al. Reduced beta 1 receptor messenger RNA abundance in the failing human heart. J Clin Invest 1993; 92:2737-45.
24. Li Y Y, Feldman A M, Sun Y, McTiernan C F. Differential expression of tissue inhibitors of metalloproteinases in the failing human heart. Circulation 1998; 98:1728-34.
25. Huang D W, Sherman B T, Tan Q et al. DAVID Bioinformatics Resources: expanded annotation database and novel algorithms to better extract biology from large gene lists. Nucleic Acids Res 2007; 35:W169-75.
26. Huang da W, Sherman B T, Lempicki R A. Systematic and integrative analysis of large gene lists using DAVID bioinformatics resources. Nat Protoc 2009; 4:44-57.
27. Su F, Myers V D, Knezevic T et al. Bcl-2-associated athanogene 3 protects the heart from ischemia/reperfusion injury. JCI Insight 2016; 1:e90931.
28. Zhou Y Y, Wang S Q, Zhu W Z et al. Culture and adenoviral infection of adult mouse cardiac myocytes: methods for cellular genetic physiology. Am J Physiol Heart Circ Physiol 2000; 279:H429-36.
29. Song J, Zhang X Q, Wang J et al. Regulation of cardiac myocyte contractility by phospholemman: Na+/Ca2+ exchange versus Na+-K+-ATPase. Am J Physiol Heart Circ Physiol 2008; 295:H1615-25.
30. Wang J, Gao E, Rabinowitz J et al. Regulation of in vivo cardiac contractility by phospholemman: role of Na+/Ca2+ exchange. Am J Physiol Heart Circ Physiol 2011; 300:H859-68.
31. Zhu W, Tilley D G, Myers V D, Coleman R C, Feldman A M. Arginine vasopressin enhances cell survival via a G protein-coupled receptor kinase 2/beta-arrestin1/extracellular-regulated kinase 1/2-dependent pathway in H9c2 cells. Mol Pharmacol 2013; 84:227-35.
32. Tilley D G, Zhu W, Myers V D et al. beta-adrenergic receptor-mediated cardiac contractility is inhibited via vasopressin type 1A-receptor-dependent signaling. Circulation 2014; 130:1800-11.
33. Cheung J Y, Thompson I G, Bonventre J V. Effects of extracellular calcium removal and anoxia on isolated rat myocytes. Am J Physiol 1982; 243:C184-90.
34. Tucker A L, Song J, Zhang X Q et al. Altered contractility and [Ca2+]i homeostasis in phospholemman-deficient murine myocytes: role of Na+/Ca2+ exchange. Am J Physiol Heart Circ Physiol 2006; 291:H2199-209.
35. Miller B A, Hoffman N E, Merali S et al. TRPM2 channels protect against cardiac ischemia-reperfusion injury: role of mitochondria. J Biol Chem 2014; 289:7615-29.
36. Kirichok Y, Krapivinsky G, Clapham D E. The mitochondrial calcium uniporter is a highly selective ion channel. Nature 2004; 427:360-4.
37. Hoffman N E, Chandramoorthy H C, Shamugapriya S et al. MICU1 motifs define mitochondrial calcium uniporter binding and activity. Cell Rep 2013; 5:1576-1588.
38. Fang X, Bogomolovas J, Trexler C, Chen J. The BAG3-dependent and -independent roles of cardiac small heat shock proteins. JCI Insight 2019; 4.
39. Tahrir F G, Knezevic T, Gupta M K et al. Evidence for the Role of BAG3 in Mitochondrial Quality Control in Cardiomyocytes. J Cell Physiol 2017; 232:797-805.
40. Liu G, Zou H, Luo T et al. Caspase-Dependent and Caspase-Independent Pathways Are Involved in Cadmium-Induced Apoptosis in Primary Rat Proximal Tubular Cell Culture. PLoS One 2016; 11:e0166823.
41. Mastrocola R, Aragno M, Alloatti G, Collino M, Penna C, Pagliaro P. Metaflammation: Tissue-Specific Alterations of the NLRP3 Inflammasome Platform in Metabolic Syndrome. Curr Med Chem 2018; 25:1294-1310.
42. Mashimo M, Bu X, Aoyama K et al. PARP1 inhibition alleviates injury in ARH3-deficient mice and human cells. JCI Insight 2019; 4.
43. Romero-Becerra R, Santamans A M, Folgueira C, Sabio G. p38 MAPK Pathway in the Heart: New Insights in Health and Disease. Int J Mol Sci 2020; 21.
44. Green L C, Anthony S R, Slone S et al. Human antigen R as a therapeutic target in pathological cardiac hypertrophy. JCI Insight 2019; 4.
45. Krishnamurthy P, Lambers E, Verma S et al. Myocardial knockdown of mRNA-stabilizing protein HuR attenuates post-MI inflammatory response and left ventricular dysfunction in IL-10-null mice. FASEB J 2010; 24:2484-94.
46. Mammucari C, Gherardi G, Rizzuto R. Structure, Activity Regulation, and Role of the Mitochondrial Calcium Uniporter in Health and Disease. Front Oncol 2017; 7:139.
47. Doukhanina E V, Chen S, van der Zalm E, Godzik A, Reed J, Dickman M B. Identification and functional characterization of the BAG protein family in Arabidopsis thaliana. J Biol Chem 2006; 281:18793-801.
48. Haudek S B, Taffet G E, Schneider M D, Mann D L. TNF provokes cardiomyocyte apoptosis and cardiac remodeling through activation of multiple cell death pathways. J Clin Invest 2007; 117:2692-701.
49. Zhang J, Webster J D, Dugger D L et al. Ubiquitin Ligases cIAP1 and cIAP2 Limit Cell Death to Prevent Inflammation. Cell Rep 2019; 27:2679-2689 e3.
50. Siddiqui W A, Ahad A, Ahsan H. The mystery of BCL2 family: Bcl-2 proteins and apoptosis: an update. Arch Toxicol 2015; 89:289-317.
51. Baumgartner H K, Gerasimenko J V, Thorne C et al. Caspase-8-mediated apoptosis induced by oxidative stress is independent of the intrinsic pathway and dependent on cathepsins. Am J Physiol Gastrointest Liver Physiol 2007; 293:G296-307.
52. Kruidering M, Evan G I. Caspase-8 in apoptosis: the beginning of “the end” ? IUBMB Life 2000; 50:85-90.
53. Obexer P, Ausserlechner M J. X-linked inhibitor of apoptosis protein—a critical death resistance regulator and therapeutic target for personalized cancer therapy. Front Oncol 2014; 4:197.
54. Kearney C J, Sheridan C, Cullen S P et al. Inhibitor of apoptosis proteins (IAPs) and their antagonists regulate spontaneous and tumor necrosis factor (TNF)-induced proinflammatory cytokine and chemokine production. J Biol Chem 2013; 288:4878-90.
55. Brenner D, Blaser H, Mak T W. Regulation of tumour necrosis factor signalling: live or let die. Nat Rev Immunol 2015; 15:362-74.
56. Liu T, O'Rourke B. Regulation of mitochondrial Ca2+ and its effects on energetics and redox balance in normal and failing heart. J Bioenerg Biomembr 2009; 41:127-32.
57. Maack C, Cortassa S, Aon M A, Ganesan A N, Liu T, O'Rourke B. Elevated cytosolic Na+ decreases mitochondrial Ca2+ uptake during excitation-contraction coupling and impairs energetic adaptation in cardiac myocytes. Circ Res 2006; 99:172-82.
58. Liu T, Yang N, Sidor A, O'Rourke B. MCU Overexpression Rescues Inotropy and Reverses Heart Failure by Reducing S R Ca(2+) Leak. Circ Res 2021; 128:1191-1204.
59. Tang Y, Wu Y. Decreased ATP production during mitochondrial calcium uniporter inhibition enhances autophagy and mitophagy to provide cardioprotection in cardiac failure. Int J Cardiol 2019; 282:67.
60. Yu Z, Chen R, Li M et al. Mitochondrial calcium uniporter inhibition provides cardioprotection in pressure overload-induced heart failure through autophagy enhancement. Int J Cardiol 2018; 271:161-168.
61. Langenbacher A D, Shimizu H, Hsu W et al. Mitochondrial Calcium Uniporter Deficiency in Zebrafish Causes Cardiomyopathy With Arrhythmia. Front Physiol 2020; 11:617492.
62. Suarez J, Cividini F, Scott B T et al. Restoring mitochondrial calcium uniporter expression in diabetic mouse heart improves mitochondrial calcium handling and cardiac function. J Biol Chem 2018; 293:8182-8195.
63. Garbincius J F, Elrod J W. Is the Failing Heart Starved of Mitochondrial Calcium? Circ Res 2021; 128:1205-1207.
64. Pan X, Liu J, Nguyen T et al. The physiological role of mitochondrial calcium revealed by mice lacking the mitochondrial calcium uniporter. Nat Cell Biol 2013; 15:1464-72.
65. Huang S C, Vu L V, Yu F H, Nguyen D T, Benz E J, Jr. Multifunctional protein 4.1R regulates the asymmetric segregation of Numb during terminal erythroid maturation. J Biol Chem 2021; 297:101051.

1. A method to reduce, inhibit or decrease TNF signaling comprising: administering to a patient an amount of BCL2-associated athanogene 3 (BAG3) encoding nucleic acid, BAG3 protein or BAG3 peptide thereby reducing, inhibiting or decreasing TNF signaling.

2. A method for treating a patient suffering from, or, at risk of developing inflammation comprising: administering to the patient an amount of an agent that modulates expression or amount of BCL2-associated athanogene 3 (BAG3) encoding nucleic acid, BAG3 protein or BAG3 peptide thereby treating inflammation.

3. A method to reduce, inhibit or decrease inflammation or inflammatory response comprising: administering to a patient an amount of BCL2-associated athanogene 3 (BAG3) encoding nucleic acid, BAG3 protein or BAG3 peptide thereby reducing, inhibiting, or decreasing inflammation or an inflammatory response.

4. The method of claim 1, wherein the TNF signaling occurs in the pulmonary system, lung, cardiovascular system, central nervous system, bone, skeletal joints, skeletal muscle, gastrointestinal system, stomach, small intestine, large intestine, liver, kidney or pancreas.

5. The method of claim 2, wherein the inflammation or inflammatory response affects the pulmonary system, lung, cardiovascular system, central nervous system, bone, skeletal joints, skeletal muscle, gastrointestinal system, stomach, small intestine, large intestine, liver, kidney or pancreas.

6. The method claim 2, wherein the inflammation or inflammatory response comprises chronic inflammatory disease, chronic inflammatory demyelinating polyneuropathy, primary immune thrombocytopenia, geriatric anorexia, gut inflammation, inflammatory bowel disease, ulcerative colitis, Crohn's disease, lupus, rheumatoid arthritis, chronic myocarditis, chronic myocarditis after Covid 19 infection, psoriasis, psoriatic arthritis or ankylosing spondylitis.

7.-10. (canceled)

11. The method of claim 1, wherein the BAG3 encoding nucleic acid comprises an expression vector expressing a BAG3 protein or active BAG3 peptide thereof.

12. The method of claim 11, wherein the expression vector further comprises a promoter, the promoter comprising an inducible promoter, a constitutive promoter, bicistronic promoter, tissue specific promoter or cardiac specific promoter.

13. The method of claim 11, wherein the expression vector comprises a viral vector, cardiotropic vector, plasmid, or a yeast vector.

14. The method of claim 13, wherein a viral or cardiotropic vector comprises an adenovirus vector, an adeno-associated virus vector (AAV), a coxsackie virus vector, cytomegalovirus vector, Epstein-Barr virus vector, parvovirus vector, or hepatitis virus vectors.

15. The method of claim 14, wherein the AAV vector comprises a capsid protein having 90% or more sequence identity to any of AAV1, AAV2, AAV3, AAV4, AAV5, AAV6, AAV7m AAV8, AAV9, AAV10, AAV11 or AAV12.

16. (canceled)

17.-18. (canceled)

19. The method of claim 1, wherein the patient expresses lower than normal levels of BAG3 in a tissue or organ or does not detectably express or produce functional BAG3.

20. (canceled)

21. The method of claim 11, wherein the expression vector further comprises a promoter, the promoter optionally comprising an inducible promoter, a constitutive promoter, bicistronic promoter or tissue specific promoter.

22. The method of claim 21, wherein the promoter confers expression in the pulmonary system, lung, cardiovascular system, central nervous system, bone, skeletal joints, skeletal muscle, gastrointestinal system, stomach, small intestine, large intestine, liver, kidney or pancreas.

23. The method of claim 11, wherein the expression vector further comprises an AAV inverted terminal repeat (ITR).

24. (canceled)

25. The method of claim 1, wherein the patient is human.

26. The method of claim 1, wherein the patient or human has a mutation in their endogenous BAG3 polynucleotide or polypeptide.

27. (canceled)

28. The method of claim 13, wherein the viral vector is administered or formulated at a dose from about 0.1×10¹² vector genomes (vg)/weight of the patient in kilograms (vg/kg) to about 1.0×10¹⁴ vg/kg.

29.-33. (canceled)
